SEQUENCE LISTING (24 sequences)

SEQ ID NO: 1 - protein, 205 aa, Mus musculus
GPLGSSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 2 - protein, 201 aa, homo sapiens
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 3 - protein, 203 aa, Mus musculus
MSQREGSLEDHQTDSSISFLPHLEAKIRQTHNLARLLTKYAEQLLEEYVQQQGEPFGLPGFSPPRLPLAGLSGPAPSHAGLPVSERLRQDAAALSVLPALLDAVRRRQAELNPRAPRLLRSLEDAARQVRALGAAVETVLAALGAAARGPGPEPVTVATLFTANSTAGIFSAKVLGFHVCGLYGEWVSRTEGDLGQLVPGGVA

SEQ ID NO: 4 - protein, 5 aa, Mus musculus
GPLGS

SEQ ID NO: 5 - protein, 6 aa, Mus musculus
  misc_feature (6)..(6): Xaa can be any naturally occurring amino acid
GPLGSX

SEQ ID NO: 6 - protein, 103 aa, Mus musculus
GPLGSSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPP

SEQ ID NO: 7 - protein, 99 aa, Mus musculus
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPP

SEQ ID NO: 8 - protein, 103 aa, Mus musculus
GPLGSVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 9 - protein, 98 aa, Mus musculus
VCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 10 - protein, 154 aa, Mus musculus
GPLGSSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGP

SEQ ID NO: 11 - protein, 150 aa, Mus musculus
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGP

SEQ ID NO: 12 - protein, 158 aa, Mus musculus
GPLGSVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 13 - protein, 153 aa, Mus musculus
VQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 14 - protein, 107 aa, Mus musculus
GPLGSVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGP

SEQ ID NO: 15 - protein, 102 aa, Mus musculus
VQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGP

SEQ ID NO: 16 - protein, 52 aa, Mus musculus
GPLGSVCRRQAELNPRAPRLLRRLEDAARQARALGAAVEALLAALGAANRGP

SEQ ID NO: 17 - protein, 47 aa, Mus musculus
VCRRQAELNPRAPRLLRRLEDAARQARALGAAVEALLAALGAANRGP

SEQ ID NO: 18 - protein, 56 aa, Mus musculus
GPLGSRAEPPAATASAASATGVFPAKVLGLRVCGLYREWLSRTEGDLGQLLPGGSA

SEQ ID NO: 19 - protein, 51 aa, Mus musculus
RAEPPAATASAASATGVFPAKVLGLRVCGLYREWLSRTEGDLGQLLPGGSA

SEQ ID NO: 20 - protein, 100 aa, Mus musculus
MSQREGSLEDHQTDSSISFLPHLEAKIRQTHNLARLLTKYAEQLLEEYVQQQGEPFGLPGFSPPRLPLAGLSGPAPSHAGLPVSERLRQDAAALSVLPAL

SEQ ID NO: 21 - protein, 150 aa, Mus musculus
MSQREGSLEDHQTDSSISFLPHLEAKIRQTHNLARLLTKYAEQLLEEYVQQQGEPFGLPGFSPPRLPLAGLSGPAPSHAGLPVSERLRQDAAALSVLPALLDAVRRRQAELNPRAPRLLRSLEDAARQVRALGAAVETVLAALGAAARGP

SEQ ID NO: 22 - protein, 100 aa, Mus musculus
VRRRQAELNPRAPRLLRSLEDAARQVRALGAAVETVLAALGAAARGPGPEPVTVATLFTANSTAGIFSAKVLGFHVCGLYGEWVSRTEGDLGQLVPGGVA

SEQ ID NO: 23 - protein, 150 aa, Mus musculus
EPFGLPGFSPPRLPLAGLSGPAPSHAGLPVSERLRQDAAALSVLPALLDAVRRRQAELNPRAPRLLRSLEDAARQVRALGAAVETVLAALGAAARGPGPEPVTVATLFTANSTAGIFSAKVLGFHVCGLYGEWVSRTEGDLGQLVPGGVA

SEQ ID NO: 24 - protein, 100 aa, Mus musculus
QQGEPFGLPGFSPPRLPLAGLSGPAPSHAGLPVSERLRQDAAALSVLPALLDAVRRRQAELNPRAPRLLRSLEDAARQVRALGAAVETVLAALGAAARGP

(19) United States
(12) Patent Application Publication
(10) Pub. No.: US 2009/0042795 A1
(43) Pub. Date: Feb. 12, 2009

(54) Cardiac stem cell proliferation proteins, fragments thereof and methods of modulating stem cell proliferation and differentiation

(76) Inventors: Pasan Fernando, Ottawa (CA); Lynn Megeney, Ottawa (CA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/156,953

(22) Filed: Jun. 5, 2008

Related U.S. Application Data
(60) Provisional application No. 60/933,196, filed on Jun. 5, 2007.

(51) Int. Cl. C07K 14/46; C12N 15/11; C12N 15/63; A61K 38/17; A61P 9/00

(52) U.S. Cl. 514/12; 530/350; 536/23.1; 435/320.1

(57) ABSTRACT

The present invention provide cardiac stem cell proliferation proteins and fragments thereof that promote proliferation and/or differentiation of cardiac stem cells. Also provided are methods of treating subjects with heart disease or defects, the methods comprising administering a cardiac stem cell proliferation protein or a fragment thereof to the subject in need of such treatment.

### RELATED APPLICATIONS

This application claims the benefit of U.S. Provisional Application No. 60/933,196, filed on Jun. 5, 2007, the entire contents of which are hereby incorporated herein by reference.

### FIELD OF THE INVENTION

The present invention relates to cardiac stem cell proliferation proteins, fragments thereof and methods of modulating stem cell proliferation and differentiation. More specifically, the present invention relates to cardiac stem cell proliferation proteins, fragments thereof and methods of modulating stem cell proliferation and differentiation in cardiac tissue.

### BACKGROUND OF THE INVENTION

Stem cells are undifferentiated, or immature, cells that are capable of giving rise to multiple, specialised cell types and ultimately to terminally differentiated cells. Unlike any other cells, they are able to renew themselves such that essentially an endless supply of mature cell types can be generated when needed. Due to this capacity for self-renewal, stem cells are therapeutically useful for the regeneration and repair of tissues.

Stem cells have the potential for providing benefits in a variety of clinical settings. However, their use has been limited in part due to difficulties in obtaining a sufficient number of target cells and stimulating terminal differentiation of these stem cells into mature tissue specific cells.

Adult bone marrow and many other somatic tissues are believed to contain a population of pluripotent stem cells that can differentiate into cells of various phenotypes. For example, a multi-potent stem cell-like population (SP) within the adult murine heart has been reported (Hierlihy, A. M., et al., (2002) FEBS Letters, 530:239-243). Under circumstances of attenuated growth, these cells become activated and differentiate into cardiomyocytes. Methods of repairing or regenerating damaged myocardium by administration of somatic stem cells, derived from cardiac or other tissues, and cytokines, such as stem cell factor (SCF), granulocyte-colony stimulating factor (G-CSF), granulocyte-macrophage colony stimulating factor (GM-CSF), stromal cell-derived factor-1, steel factor, vascular endothelial growth factor, macrophage colony stimulating factor, granulocyte-macrophage stimulating factor or interleukin-3, have recently been described (U.S. Patent Application 20030054973).

The use of cytokines, cellular mediators that can regulate growth and differentiation, to promote differentiation of stem cells has been described. For example, U.S. Patent Application 20030027330 describes methods of producing differentiated mammalian cells or tissue from stem cells by co-culturing stem cells with developing or developed allogeneic or xenogeneic cells and optionally a cytokine, growth factor or chemokine; U.S. Patent Application 20030103951 describes a method of regenerating cardiac muscle by administration of mesenchymal stem cells, which may be genetically modified to produce proteins that are important in differentiation, such as cytokines, growth factors, myogenic factors and transcription factors, and U.S. Patent Application 20020142457 describes methods for proliferating cells having the potential to differentiate into cardiomyocytes and for regulating their differentiation into cardiomyocytes using various cytokines and transcription factors.

Therapy involving the transfer of cells derived from bone marrow has also been researched in animals and humans. A recent study (Schächinger, V., et al, (2006) NEJM, 355:1210-1221) showed a beneficial effect on cardiac function, specifically left ventricular contractile function, associated with intracoronary infusion of bone marrow cells (BMC) after percutaneous coronary intervention for acute myocardial infarction. Similarly, Assmus et al (Assmus, B, (2006) NEJM, 355:1222-1232) showed that infusion of BMC increases global cardiac function in patients having suffered myocardial infarction. However, both studies showed only a modest improvement in cardiac function. Others (Meyer, G. P., et al (2006) Circulation, 113:1287-1294) have shown that improvements observed following infusion with BMC at 6 months are no longer significant at 18 months. In yet another study, Lunde et al (Lunde, K., et al, (2006) NEJM, 355:1199-1209) found no beneficial effects on heart function in patients having received intracoronary injections of autologous BMC.

The above studies and other showing the regeneration of cardiac tissue by BMC (Orlic, D, et al, (2001) Nature, 410:701-705) provided hope for the development of cell-based therapies. However, the efficacy of BMC to generate cardiomyocytes has been questioned (Murry, C. E., et al (2004) Nature, 428:664-668; Balsam, L. B. et al, (2004) Nature, 428:668-673). Furthermore, another study has shown that only a low percentage of infused BMC are retained in the heart (Hofmann, M., et al (2005) Circulation, 111:2198-2202).

To date, modest success has been achieved in cell-based therapies in the treatment of myocardial infarction. Administration of BMC has shown some improvement in cardiac function. However, the positive results are tempered by the questionable ability of BMC to effectively generate cardiomyocytes, the low retention of infused BMC in the heart, and the lack of long-term benefits.

There is a need in the art for novel compounds and compositions that can promote cardiac stem cell proliferation and/or differentiation. Further, there is a need in the art for novel methods that may be employed to promote cardiac stem cell proliferation and/or differentiation following cardiac injury, for example, by myocardial infarction, disease, infection or the like.

### SUMMARY OF THE INVENTION

The present invention relates to cardiac stem cell proliferation proteins, fragments thereof and methods of modulating stem cell proliferation and differentiation. More specifically, the present invention is directed to cardiac stem cell proliferation proteins, fragments thereof and methods of modulating stem cell proliferation and differentiation in cardiac tissue.

According to the present invention, there is provided a cardiac stem cell proliferation protein as defined by SEQ ID NO:1, a protein having at least 70% amino acid identity to SEQ ID NO:1, 2 or 3 or

a fragment of a cardiac stem cell proliferation protein comprising
a) at least about 25 consecutive amino acids of SEQ ID NO:1, SEQ ID NO:2 or SEQ ID NO:3; or
b) a polypeptide having at least 70% amino acid identity to the fragment as defined in a), and;

wherein the cardiac stem cell proliferation protein or fragment thereof is not SEQ ID NO:2 or SEQ ID NO:3 or any other naturally occurring cardiac stem cell proliferation protein and wherein;

the cardiac stem cell proliferation protein or fragment thereof promotes proliferation or differentiation of cardiac stem cells. In a preferred embodiment, the cardiac stem cell proliferation protein or fragment thereof promotes proliferation and differentiation of cardiac stem cells.

The present invention also provides a nucleic acid encoding the cardiac stem cell proliferation protein or fragment as defined above. The nucleic acid may comprise a vector. In a preferred embodiment, the vector is an adenoviral vector capable of producing the protein or fragment in vivo, more preferably in a human.

The present invention also provides a composition comprising a cardiac stem cell proliferation protein or fragment thereof as described above and a pharmaceutically acceptable carrier, diluent or excipient.

Also provided by the present invention is a kit comprising

a) a cardiac stem cell proliferation protein, fragment thereof and/or a nucleotide sequence encoding said cardiac stem cell proliferation protein or fragment thereof; and;

b) one or more pharmaceutically acceptable carriers, diluents, or excipients;

c) one or more devices for delivering or administering said cardiac stem cell proliferation protein, fragment thereof or a nucleotide sequence encoding said cardiac stem cell proliferation protein or fragment thereof;

d) instructions for administering said cardiac stem cell proliferation protein, fragment thereof or a nucleotide sequence encoding said cardiac stem cell proliferation protein or fragment thereof, or

e) any combination or sub-combination of b-d.

Also provided by the present invention is a method of treating a subject having heart disease, injury or a heart defect, the method comprising administering a cardiac stem cell proliferation protein or a fragment thereof to the subject in at least one single continuous low dose over a period of from about 1 to about 30 days.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows the amino acid sequence of cardiac stem cell proliferation proteins including a representative cardiac stem cell proliferation protein as provided by the present invention (FIG. 1A; SEQ ID NO:1), human cardiac stem cell proliferation protein (SEQ ID NO:2) and mouse cardiac stem cell proliferation protein (SEQ ID NO:3).

FIG. 2 shows various constructs according to the present invention. The full length cardiac stem cell proliferation protein construct (SP200aFL; SEQ ID NO:3)) comprises amino acids 1-203 of SEQ ID NO:3 attached to a glutathione S-transferase (GST) domain. The N-terminal (100 amino acids) construct (SP100aN; SEQ ID NO:20) comprises amino acids 1-100 of SEQ ID NO:3 attached to a GST domain. The N-terminal (150 amino acids) construct (SP150aN; SEQ ID NO:21) comprises amino acids 1-150 of SEQ ID NO:3 attached to a GST domain. The C-terminal (100 amino acids) construct (SP100aC; SEQ ID NO:22) comprises amino acids 104-203 of SEQ ID NO:3 attached to a GST domain. The C-terminal (150 amino acids) construct (SP150aC; SEQ ID NO:23) comprises amino acids 54-203 of SEQ ID NO:3 attached to a GST domain. The middle fragment construct (SP100aM; SEQ ID NO:24) comprises amino acids 51-150 of SEQ ID NO:3 attached to a GST domain. The GST domain is cleaved from the constructs prior to use unless clearly indicated otherwise.

FIG. 3 shows a comparison of representative amino acid sequences of cardiac stem cell proliferation protein and fragments thereof according to the present invention. FIG. 3A shows the sequence of the full length murine cardiac stem cell proliferation protein encompassed by the SP200aFL (SEQ ID NO:3) construct. FIG. 3B shows the sequence of the N-terminal fragment encompassed by the SP100aN construct (SEQ ID NO:20). FIG. 3C shows the sequence of the N-terminal fragment encompassed by the SP150aN construct (SEQ ID NO:21). FIG. 3D shows the sequence of the C-terminal fragment encompassed by the SP100aC construct (SEQ ID NO:22). FIG. 3E shows the sequence of the C-terminal fragment encompassed by the SP150aC construct (SEQ ID NO:23).

FIG. 3F shows the sequence of the middle fragment encompassed by the SP100aM construct (SEQ ID NO:24).

FIG. 4 shows the purification of a GST-cardiac stem cell proliferation protein construct according to the present invention. GST-cardiac stem cell proliferation protein was expressed in bacteria and total (unpurified) lysate was analyzed by SDS-PAGE (left panel). GST-cardiac stem cell proliferation protein bacterial lysate was purified on glutathione affinity column via FPLC (centre panel). Purity of eluted protein fractions was then evaluated by SDS-PAGE (right panel).

FIG. 5 shows the in vitro pharmacokinetic analysis of murine SP200aFL in h9c2 cells. Levels of phospho-tyrosine STAT3 (STAT3pTyr) were elevated after 15 and 30 minutes of stimulation with SP200aFL, with a transient increase noted after 120 minutes. Activity of STAT3pTyr was still detected after 360 min of initial stimulation with SP200aFL.

FIG. 6 shows the activation of STAT3 signalling in porcine epithelial cells by human SP200aFL (SEQ ID NO:2). FIG. 6A shows that 1.5 μg of hSP200aFL (SEQ ID NO:2) maximally stimulates STAT3 tyrosine phosphorylation.

FIG. 6B is an analysis demonstrating that both low and high concentrations of hSP200aFL had similar effects on STAT3 serine phosphorylation (STAT3pSer).

FIG. 6C shows that the levels of total STAT3 were similar under the conditions tested.

FIG. 7 shows results of the activation of STAT3 signalling by cardiac stem cell proliferation protein fragments. FIG. 7A shows the activation of STAT3 signalling by the carboxyl-terminal fragment (SP100aC; SEQ ID NO:22) in h9c2 cells. SP100aC stimulated tyrosine phosphorylation of STAT3 in a dose dependent manner to a maximum of 1.0 μg and inhibited STAT3pTyr activity thereafter. FIG. 7B shows the activation of STAT3 signalling by the middle fragment (SP100aM; SEQ ID NO:24). FIG. 7C shows the activation of STAT3 signalling by the GST-linked C-terminal and N-terminal fragments. FIG. 7D shows the activation of STAT3 signalling by the GST-linked C-terminal fragment. FIG. 7E shows the activation of STAT3 signalling by the GST-linked middle fragment.

FIG. 8 shows the effect of a sustained low dosage of SP200aFL (SEQ ID NO:3) on rat myocardial STAT3 signalling in vivo. Western analyses of heart tissue lysates indicated the STAT3pTyr was elevated after 3 and 16 days (FIG. 8A). In addition, the levels of total STAT3 were not changed (FIG. 8B).

FIG. 9 shows results depicting STAT3 activation by adenoviral cardiac stem cell proliferation protein in porcine myocardium. STAT3pTyr was detected in both the left and right ventricle using an anti-STAT3 phosphotyrosine antibody, demonstrating that STAT3 activity can be readily detected in porcine hearts.

FIG. 10 shows results suggesting cardiac stem cell proliferation protein treatment promotes restoration of cardiomyocytes within the infarct area. Rats subject to myocardial infarct were either left untreated or treated with adenovirus expressing cardiac stem cell proliferation protein and allowed to recover for 8 weeks. The hearts from each animal were excised, fixed in formalin, embedded in paraffin and sectioned. The sections were de-paraffinized and treated with 5% horse serum with 3% Triton-X in PBS for 1 h. The sections were incubated overnight with anti-adult myosin heavy chain (MHC) or anti-embryonic myosin heavy chain diluted in PBS. The sections were washed and incubated with secondary antibody for 1 h and visualized with diaminobenzidine tetrahydrochloride (DAB) reagent. A greater number of embryonic myosin heavy chain positive cells were present in hearts treated with adenovirus expressing cardiac stem cell proliferation protein suggesting the creation of new cardiomyocytes.

FIG. 11 shows results suggesting cardiac stem cell proliferation protein therapy reduces scar tissue formation after infarct.

FIG. 12 shows results suggesting cardiac stem cell proliferation protein therapy improves cardiac function after infarct.

FIG. 13 shows a graphical depiction of cardiac stem cell proliferation protein plasma kinetics in rat. Following a baseline blood draw, a single systemic dose of murine cardiac stem cell proliferation protein (25 ng/g) was delivered into rats. Subsequent blood draws were taken at the time points indicated. An ELISA was used to measure plasma concentrations of murine cardiac stem cell proliferation protein in rat samples. In the graph shown, cardiac stem cell proliferation protein concentrations spiked initially after 1 day and then gradually declined toward baseline.

FIG. 14 shows a graphical depiction comparing plasma kinetics in rat using adenoviral delivery of cardiac stem cell proliferation protein and protein pump delivery methods. Plasma was collected from animals injected with either adenovirus expressing cardiac stem cell proliferation protein or cardiac stem cell proliferation protein delivered by Alzet mini-osmotic pumps, and analyzed by ELISA. A steady increase in the concentration of cardiac stem cell proliferation protein was observed in rats injected with adenovirus expressing cardiac stem cell proliferation protein. After 8-10 days, the concentration of cardiac stem cell proliferation protein remained elevated at approximately 8 ng/ml. Rats receiving cardiac stem cell proliferation protein via the osmotic pumps reached plasma levels that were slightly lower than adenovirus expressing cardiac stem cell proliferation protein treated animals; however, a gradual decrease in cardiac stem cell proliferation protein concentration was observed after approximately 8 days.

FIG. 15 shows a graphical depiction of the plasma kinetics of human cardiac stem cell proliferation protein in pig by an adenoviral delivery system. Pigs were injected with adenovirus expressing cardiac stem cell proliferation protein (7.5×10⁶ viral particles/g body weight) and blood samples were collected at the indicated time points. Pig plasma was analyzed by ELISA for human cardiac stem cell proliferation protein. An increase in cardiac stem cell proliferation protein concentration was observed within 2 days following injection and a gradual decrease was noted thereafter.

FIG. 16 shows results of infarct volume as a function of time following administration of adenovirus expressing cardiac stem cell proliferation protein or control. Myocardial infarct was induced using a 7 French Sheath balloon catheter that was inserted into the right femoral artery. The balloon was expanded for 90 minutes and then released. Two of the pigs received adenovirus expressing cardiac stem cell proliferation protein delivered as a 100 cc infusion through the ear vein immediately after the infarct. A third pig received adenoviral cardiac stem cell proliferation protein in a similar manner but at 48 h after the infarct. In all animals, a baseline MRI scan was performed at 48 h following the infarct and a second infusion of adenovirus expressing cardiac stem cell proliferation protein was administered at 2 weeks following the infarct. The animals were survived to 5-6 weeks with a final MRI scan prior to sacrifice. MRI analysis of infarct volume revealed that animals receiving adenovirus expressing cardiac stem cell proliferation protein had an approximate 21% reduction in infarct volume compared to a 6% reduction in control animals. This data indicates that adenovirus expressing cardiac stem cell proliferation protein treated animals had a smaller infarct size with less scar tissue formation.

FIG. 17 shows results of left ventricular mass as a function of time following administration of adenovirus expressing cardiac stem cell proliferation protein or control. An elevation in LV mass was noted in both animals treated with adenovirus expressing cardiac stem cell proliferation protein and control animals. Although an interim increase in LV mass is normally associated with cardiac hypertrophy, the animals in this study started at a body weight of 25 kg and end at approximately 70-80 kg. Therefore it is not unreasonable to expect mass-compensated increases in organ size.

FIG. 18 shows results of left ventricular ejection fraction as a function of time following administration of adenovirus expressing cardiac stem cell proliferation protein or control. The LVEF in pigs treated with adenovirus expressing cardiac stem cell proliferation protein improved dramatically (12% absolute increase and 52% relative increase). This was in sharp contrast to control animals that had a 1% absolute decrease and a 4% relative decrease. This data strongly support a positive role for cardiac stem cell proliferation protein in improving cardiac function following myocardial infarct.

### DESCRIPTION OF PREFERRED EMBODIMENT

The present invention relates to cardiac stem cell proliferation proteins, fragments thereof, and methods of modulating stem cell proliferation and differentiation. More specifically, the present invention relates to cardiac stem cell proliferation proteins, fragments thereof and methods of modulating stem cell proliferation and differentiation in cardiac tissue.

Interruption of the blood flow to the heart leads to myocardial infarction, commonly known as a heart attack. This starves the cardiac muscle cells of oxygen, leading to death of the heart tissue. It has been shown by others that regeneration of cardiac muscles is possible (see for example, U.S. Patent Application 20020142457 and U.S. Patent Application 20030103951). The results provided herein suggest that cardiac stem cell proliferation proteins and fragments thereof can be used to promote activation and/or differentiation of cardiac stem cells into cardiomyocytes, thus promoting the repair or regeneration of damaged cardiac tissue.

The following description is of a preferred embodiment.

According to the present invention, there is provided a cardiac stem cell proliferation protein or a fragment of a cardiac stem cell proliferation protein, the fragment comprising at least 25 amino acids, and exhibiting at least one or more biological activities of a naturally occurring cardiac stem cell proliferation protein. In a preferred embodiment, the present invention provides a cardiac stem cell proliferation protein as defined by SEQ ID NO:1, a protein having at least 70% amino acid identity to SEQ ID NOs:1, 2 or 3, or

a fragment of a cardiac stem cell proliferation protein comprising
a) at least about 25 consecutive amino acids of SEQ ID NO:1, SEQ ID NO:2 or SEQ ID NO:3; or
b) a polypeptide having at least 70% amino acid identity to the fragment as defined in a); and;

wherein the cardiac stem cell proliferation protein or fragment thereof is not SEQ ID NO:2 or SEQ ID NO:3 or any other naturally occurring cardiac stem cell proliferation protein and wherein;

the cardiac stem cell proliferation protein or fragment thereof promotes proliferation or differentiation of cardiac stem cells. In a preferred embodiment, the cardiac stem cell proliferation protein or fragment thereof promotes proliferation and differentiation of cardiac stem cells.

Specific cardiac stem cell proliferation proteins or fragments thereof contemplated by the present invention include, without limitation:

(SEQ ID NO: 1)

GPLGSSRREGSLEDPQTDSSVSLLPHLEAKIRQTHSLAHLLTKYAEQLLQ
EYVQLQGDPFGLPSFSPPRLPVAGLSAPAPSHAGLPVHERLRLDAAALAA
LPPLLDAVCRRQAELNPRAPRLLRRLEDAARQARALGAAVEALLAALGAA
NRGPRAEPPAATASAASATGVFPAKVLGLRVCGLYREWLSRTEGDLGQLL
PGGSA

(SEQ ID NO: 6)

GPLGSSRREGSLEDPQTDSSVSLLPHLEAKIRQTHSLAHLLTKYAEQLLQ
EYVQLQGDPFGLPSFSPPRLPVAGLSAPAPSHAGLPVHERLRLDAAALAA
LPP;

(SEQ ID NO: 7)

MSRREGSLEDPQTDSSVSLLPHLEAKIRQTHSLAHLLTKYAEQLLQEYVQ
LQGDPFGLPSFSPPRLPVAGLSAPAPSHAGLPVHERLRLDAAALAALPP;

(SEQ ID NO: 8)

GPLGSVCRRQAELNPRAPRLLRRLEDAARQARALGAAVEALLAALGAANR
GPRAEPPAATASAASATGVFPAKVLGLRVCGLYREWLSRTEGDLGQLLPG
GSA;

(SEQ ID NO: 9)

VCRRQAELNPRAPRLLRRLEDAARQARALGAAVEALLAALGAANRGPRAE
PPAATASAASATGVFPAKVLGLRVCGLYREWLSRTEGDLGQLLPGGSA;

(SEQ ID NO: 10)

GPLGSSRREGSLEDPQTDSSVSLLPHLEAKIRQTHSLAHLLTKYAEQLLQ
EYVQLQGDPFGLPSFSPPRLPVAGLSAPAPSHAGLPVHERLRLDAAALAA
LPPLLDAVCRRQAELNPRAPRLLRRLEDAARQARALGAAVEALLAALGAA
NRGP;

(SEQ ID NO: 11)

MSRREGSLEDPQTDSSVSLLPHLEAKIRQTHSLAHLLTKYAEQLLQEYVQ
LQGDPFGLPSFSPPRLPVAGLSAPAPSHAGLPVHERLRLDAAALAALPPL
LDAVCRRQAELNPRAPRLLRRLEDAARQARALGAAVEALLAALGAANRG
P;

(SEQ ID NO: 12)

GPLGSVQLQGDPFGLPSFSPPRLPVAGLSAPAPSHAGLPVHERLRLDAAA
LAALPPLLDAVCRRQAELNPRAPRLLRRLEDAARQARALGAAVEALLAAL
GAANRGPRAEPPAATASAASATGVFPAKVLGLRVCGLYREWLSRTEGDLG
QLLPGGSA;

(SEQ ID NO: 13)

VQLQGDPFGLPSFSPPRLPVAGLSAPAPSHAGLPVHERLRLDAAALAALP
PLLDAVCRRQAELNPRAPRLLRRLEDAARQARALGAAVEALLAALGAANR
GPRAEPPAATASAASATGVFPAKVLGLRVCGLYREWLSRTEGDLGQLLPG
GSA;

(SEQ ID NO: 14)

GPLGSVQLQGDPFGLPSFSPPRLPVAGLSAPAPSHAGLPVHERLRLDAAA
LAALPPLLDAVCRRQAELNPRAPRLLRRLEDAARQARALGAAVEALLAAL
GAANRGP;

(SEQ ID NO: 15)

VQLQGDPFGLPSFSPPRLPVAGLSAPAPSHAGLPVHERLRLDAAALAALP
PLLDAVCRRQAELNPRAPRLLRRLEDAARQARALGAAVEALLAALGAANR
GP;

(SEQ ID NO: 16)

GPLGSVCRRQAELNPRAPRLLRRLEDAARQARALGAAVEALLAALGAANR
GP;

(SEQ ID NO: 17)

VCRRQAELNPRAPRLLRRLEDAARQARALGAAVEALLAALGAANRGP;

(SEQ ID NO: 18)

GPLGSRAEPPAATASAASATGVFPAKVLGLRVCGLYREWLSRTEGDLGQL
LPGGSA;

(SEQ ID NO: 19)

RAEPPAATASAASATGVFPAKVLGLRVCGLYREWLSRTEGDLGQLLPGGS
A;

(SEQ ID NO: 20)

MSQREGSLEDHQTDSSISFLPHLEAKIRQTHNLARLLTKYAEQLLEEYVQ
QQGEPFGLPGFSPPRLPLAGLSGPAPSHAGLPVSERLRQDAAALSVLPA
L;

(SEQ ID NO: 21)

MSQREGSLEDHQTDSSISFLPHLEAKIRQTHNLARLLTKYAEQLLEEYVQ
QQGEPFGLPGFSPPRLPLAGLSGPAPSHAGLPVSERLRQDAAALSVLPAL
LDAVRRRQAELNPRAPRLLRSLEDAARQVRALGAAVETVLAALGAAARG
P;

(SEQ ID NO: 22)

VRRRQAELNPRAPRLLRSLEDAARQVRALGAAVETVLAALGAAARGPGPE
PVTVATLFTANSTAGIFSAKVLGFHVCGLYGEWVSRTEGDLGQLVPGGV
A;

(SEQ ID NO: 23)

EPFGLPGFSPPRLPLAGLSGPAPSHAGLPVSERLRQDAAALSVLPALLDA
VRRRQAELNPRAPRLLRSLEDAARQVRALGAAVETVLAALGAAARGPGPE
PVTVATLFTANSTAGIFSAKVLGFHVCGLYGEWVSRTEGDLGQLVPGGV
A,
and

(SEQ ID NO: 24)

QQGEPFGLPGFSPPRLPLAGLSGPAPSHAGLPVSERLRQDAAALSVLPAL
LDAVRRRQAELNPRAPRLLRSLEDAARQVRALGAAVETVLAALGAAARG
P.

Other cardiac stem cell proliferation proteins and fragments thereof for example, but not limited as described herein and throughout are also contemplated by the present invention, but are not meant to be limiting in any manner.

A fragment of cardiac stem cell proliferation protein as defined above comprises at least about 25 amino acids. However, the present invention contemplates fragments that comprise about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 amino acids or any number therein between. Further, the fragments of cardiac stem cell proliferation protein may be defined as comprising a range of sizes encompassed by any two of the values listed above, or any two values therein between. In a specific, non-limiting example, the fragment may comprise at least about 50, 100, or 150 consecutive amino acids of a cardiac stem cell proliferation protein.

The cardiac stem cell proliferation protein may include polypeptides that are substantially identical to SEQ ID NO:1 or a naturally occurring cardiac stem cell proliferation protein (for example SEQ ID NOs: 2 or 3), and provided that such sequences exhibit at least one biological activity as described herein. Sequences are considered “substantially identical” when at least about 70% or more of the amino acids match over a defined length of the polypeptide sequence. For example, the cardiac stem cell proliferation protein as defined by the present invention may be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or greater identical over a defined length. Further, the cardiac stem cell proliferation protein provided by the present invention may be defined as comprising a range of sequence identity as defined by any two of the values listed or any values therein between.

Also, as described above, it is contemplated that the fragment of cardiac stem cell proliferation protein may include polypeptides that are substantially identical to a consecutive amino acid sequence from a cardiac stem cell proliferation protein (for example, SEQ ID NO:1) or a naturally occurring cardiac stem cell proliferation protein (for example SEQ ID NOs: 2 or 3), and provided that such sequences exhibit at least one biological activity as described herein. Sequences are considered “substantially identical” when at least about 70% or more of the amino acids match over a defined length of the polypeptide sequence. For example, the fragments of cardiac stem cell proliferation protein as defined by the present invention may be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9% or 100% identical over a defined length. Further, the fragment of cardiac stem cell proliferation protein provided by the present invention may be defined as comprising a range of sequence identity as defined by any two of the values listed or any values therein between.

In an embodiment of the present invention, but without wishing to be limiting in any manner, the fragment of cardiac stem cell proliferation protein is a C-terminal fragment, an N-terminal fragment, or an internal fragment of SEQ ID NO:1, 2 or 3. In a further embodiment, but not wishing to be limiting in any manner, the cardiac stem cell proliferation protein or fragment thereof comprises the N-terminal amino acid sequence GPLGS (SEQ ID NO:4). In yet a further non-limiting embodiment, the stem cell proliferation protein or fragment thereof comprises GPLGSX (SEQ ID NO:5) wherein X is any amino acid except methionine.

Any method known in the art may be used for determining the degree of identity between polypeptides sequences. For example, but without wishing to be limiting, a sequence search method such as BLAST (Basic Local Alignment Search Tool; (Altschul S F, Gish W, Miller W, Myers E W, Lipman D J (1990) J Mol Biol 215, 403 410) can be used according to default parameters as described by Tatiana et al., FEMS Microbial Lett. 174:247 250 (1999), or on the National Center for Biotechnology Information web page at ncbi.nlm.gov/BLAST/, for searching closely related sequences. BLAST is widely used in routine sequence alignment; modified BLAST algorithms such as Gapped BLAST, which allows gaps (either insertions or deletions) to be introduced into alignments, or PSI-BLAST, a sensitive search for sequence homologs (Altschul et al., Nucleic Acids Res. 25:3389 3402 (1997); or FASTA, which is available on the world wide web at ExPASy (EMBL—European Bioinformatics Institute).

The cardiac stem cell proliferation protein or fragment thereof also includes derivatives of the polypeptides described above. A “derivative” is a polypeptide or peptide containing additional chemical or biochemical moieties not normally a part of a naturally occurring protein sequence. Derivatives include polypeptides and peptides in which the amino-terminus and/or the carboxy-terminus and/or one or more amino acid side chain has been modified with a desired chemical substituent group, as well as cyclic, polypeptides and peptides, polypeptides and peptides fused to other heterologous proteins or carriers, glycosylated or phosphorylated polypeptides and peptides, polypeptides and peptides conjugated to lipophilic moieties (for example, caproyl, lauryl, stearoyl moieties) and polypeptides and peptides conjugated to an antibody or other biological ligand.

Examples of chemical substituent groups that may be used to derivatise polypeptides and peptides include, but are not limited to, alkyl, cycloalkyl and aryl groups; acyl groups, including alkanoyl and aroyl groups; esters; amides; halogens; hydroxyls; carbamyls, and the like. The substituent group may also be a blocking group such as Fmoc (fluorenylmethyl-O—CO—), carbobenzoxy (benzyl-O—CO—), monomethoxysuccinyl, naphthyl-NH—CO—, acetylamino-caproyl and adamantyl-NH—CO—. Other derivatives include C-terminal hydroxymethyl derivatives, O-modified derivatives (for example, C-terminal hydroxymethyl benzyl ether) and N-terminally modified derivatives including substituted amides such as alkylamides and hydrazides.

The present invention also contemplates cardiac stem cell proliferation proteins that are multimers (ie. comprising at least one repeating unit) of a cardiac stem cell proliferation protein or fragment thereof. For example, but not to be considered limiting in any manner, the present invention comprises polypeptides that comprise multimers of at least a 25 amino acid fragment of a cardiac stem cell proliferation protein. The protein or fragments thereof may be attached directly to one another, for example, through peptide bonds, or the proteins/fragments may be attached together via a linking moiety which may comprise one or more amino acids or polypeptides, or a non-polypeptide moiety. A variety of linkers as known in the art may be employed for this purpose.

A cardiac stem cell proliferation protein or fragment thereof may be a multimer comprising between about 2 and about 10 repeating subunits, for example, 2, 3, 4, 5, 6, 7, 8, 9, or 10 repeating subunits. However, more than 10 repeating units also may be present in the multimer, if desired.

The cardiac stem cell proliferation protein or fragment thereof exhibits at least one or more biological activities of the naturally occurring cardiac stem cell proliferation protein, for example, but not limited to STAT3 tyrosine phosphorylation, a hallmark of the activation pathway. Without wishing to be bound by theory or limiting in any manner, the activation of STAT3 tyrosine phosphorylation alone or in combination with other molecular pathways is thought to be required for proliferation and/or differentiation of cardiac stem cells. In this regard, the cardiac stem cell proliferation protein or fragments thereof promote repair of damaged cardiac muscle tissue when they are administered to a subject having suffered cardiac damage.

The cardiac stem cell proliferation protein or fragments thereof can be prepared by any suitable method known in the art. For example, but without wishing to be limiting, the protein or fragments thereof may be purified from cell extracts using recombinant techniques. Shorter sequences can also be chemically synthesised by methods known in the art including, but not limited to, exclusive solid phase synthesis, partial solid phase synthesis, fragment condensation or classical solution synthesis (Merrifield (1963) J. Am. Chem. Soc. 85:2149; Merrifield (1986) Science 232:341). As described above, cardiac stem cell proliferation protein sequences or fragments thereof may be fused (either genetically or chemically) to produce longer polypeptides, for example cardiac stem cell proliferation protein multimers or multimers comprising fragments thereof. The polypeptides of the present invention may be purified using standard techniques such as, for example, but not limited to chromatography (e.g. ion exchange, affinity, size exclusion chromatography, or high performance liquid chromatography (HPLC)), centrifugation, differential solubility, or by any other suitable technique familiar to a worker skilled in the art.

The cardiac stem cell proliferation protein or fragment thereof also may be produced by recombinant techniques. Typically, this involves transformation (including one or more of transfection, transduction, and/or infection) of a suitable host cell with an expression vector comprising a polynucleotide encoding the protein or polypeptide.

The cardiac stem cell proliferation protein or fragment thereof may be fused to a heterologous protein or polypeptide sequence. The production of cardiac stem cell proliferation protein or fragments thereof as fusion proteins may simplify or improve protein purification, or may facilitate detection of the polypeptide. For example, but without wishing to be limiting in any manner, the fusion protein may be an immunoglobulin Fc domain. In such a case, the resultant cardiac stem cell proliferation protein or fragment fusion protein may be readily purified using a protein A column. In another non-limiting example, the cardiac stem cell proliferation protein or fragment thereof may be fused to glutathione S-transferase (GST) and the fusion protein purified on a glutathione column. Other non-limiting examples of fusion domains include histidine tags (purification on Ni²⁺ resin columns), a FLAG-tag (purification by anti-FLAG affinity chromatography), or to biotin (purification on streptavidin columns or with streptavidin-labelled magnetic beads). As would be readily recognized by a person of skill in the art, a linker (or “spacer”) peptide or other chemical linker may be added between the cardiac stem cell proliferation protein or fragment thereof and the fusion domain to ensure that the proteins fold independently. Once the fusion protein has been purified, the fusion domain may be removed by site-specific cleavage using a suitable chemical or enzymatic method known in the art.

The present invention also includes nucleic acids encoding cardiac stem cell proliferation protein or fragments thereof as described above. These nucleic acids also include sequences that are substantially identical to the nucleic acids, a complement of the nucleic acids, or a sequence that hybridises to the nucleic acids as defined herein under stringent hybridisation conditions, and encode a polypeptide that exhibits at least one biological activity of cardiac stem cell proliferation protein, for example, but not limited to STAT3 phosphorylation. Sequences are considered “substantially identical” when at least about 70% to 100%, for example 70%, 75%, 80%, 85%, 90%, 95%, 96%, 97%, 98%, 99%, 99.9% or 100% of the nucleotides match over a defined length of the nucleotide sequence. Preferably, sequences that are substantially identical exhibit at least about 80% and most preferably at least about 90% sequence identical over a defined length of the molecule.

Sequence similarity or identity may be determined using a nucleotide sequence comparison program, such as that provided within DNASIS (using, for example but not limited to, the following parameters: GAP penalty 5, #of top diagonals 5, fixed GAP penalty 10, k-tuple 2, floating gap 10, and window size 5). However, other methods of alignment of sequences for comparison are well-known in the art for example the algorithms of Smith & Waterman ((1981) Adv. Appl. Math., 2:482), Needleman & Wunsch ((1970) J. Mol. Biol., 48:443), Pearson & Lipman ((1988) Proc. Nat'l. Acad. Sci. USA, 85:2444), and by computerized implementations of these algorithms (GAP, BESTFIT, FASTA, and BLAST, available through the NIH.), or by manual alignment and visual inspection (see, e.g., Current Protocols in Molecular Biology, Ausubel et al., eds. 1995 supplement), or using Southern or Northern hybridization under stringent conditions (see Maniatis et al., in Molecular Cloning (A Laboratory Manual), Cold Spring Harbor Laboratory, 1982).

Stringent hybridization conditions may be, for example but not limited to hybridization overnight (from about 16-20 hours) hybridization in 4×SSC at 65° C., followed by washing in 0.1×SSC at 65° C. for an hour, or 2 washes in 0.1×SSC at 65° C. each for 20 or 30 minutes. Alternatively, an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours); or hybridization in Church aqueous phosphate buffer (7% SDS; 0.5M NaPO₄ buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each for unique sequence regions.

The present invention is further directed to a nucleotide construct comprising a nucleic acid encoding a cardiac stem cell proliferation protein or fragment thereof, as described above, operatively linked to one or more regulatory elements or regulatory regions. By “regulatory element” or “regulatory region”, it is meant a portion of nucleic acid typically, but not always, upstream of a gene, and may be comprised of either DNA or RNA, or both DNA and RNA. Regulatory elements may include those which are capable of mediating organ specificity, or controlling developmental or temporal gene activation. Furthermore, “regulatory element” includes promoter elements, core promoter elements, elements that are inducible in response to an external stimulus, elements that are activated constitutively, or elements that decrease or increase promoter activity such as negative regulatory elements or transcriptional enhancers, respectively. By a nucleotide sequence exhibiting regulatory element activity it is meant that the nucleotide sequence when operatively linked with a coding sequence of interest functions as a promoter, a core promoter, a constitutive regulatory element, a negative element or silencer (i.e. elements that decrease promoter activity), or a transcriptional or translational enhancer.

By “operatively linked” it is meant that the particular sequences, for example a regulatory element and a coding region of interest, interact either directly or indirectly to carry out an intended function, such as mediation or modulation of gene expression. The interaction of operatively linked sequences may, for example, be mediated by proteins that interact with the operatively linked sequences.

Regulatory elements as used herein, also includes elements that are active following transcription initiation or transcription, for example, regulatory elements that modulate gene expression such as translational and transcriptional enhancers, translational and transcriptional repressors, and mRNA stability or instability determinants. In the context of this disclosure, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns, or 3′ of the coding region sequence may also contribute to the regulation of expression of a coding region of interest. A regulatory element may also include those elements located downstream (3′) to the site of transcription initiation, or within transcribed regions, or both. In the context of the present invention a post-transcriptional regulatory element may include elements that are active following transcription initiation, for example translational and transcriptional enhancers, translational and transcriptional repressors, and mRNA stability determinants.

The regulatory elements, or fragments thereof, may be operatively associated (operatively linked) with heterologous regulatory elements or promoters in order to modulate the activity of the heterologous regulatory element. Such modulation includes enhancing or repressing transcriptional activity of the heterologous regulatory element, modulating post-transcriptional events, or both enhancing/repressing transcriptional activity of the heterologous regulatory element and modulating post-transcriptional events. For example, one or more regulatory elements, or fragments thereof, may be operatively associated with constitutive, inducible, tissue specific promoters or fragment thereof, or fragments of regulatory elements, for example, but not limited to TATA or GC sequences may be operatively associated with the regulatory elements of the present invention, to modulate the activity of such promoters within plant, insect, fungi, bacterial, yeast, or animal cells.

There are several types of regulatory elements, including those that are developmentally regulated, inducible and constitutive. A regulatory element that is developmentally regulated, or controls the differential expression of a gene under its control, is activated within certain organs or tissues of an organ at specific times during the development of that organ or tissue. However, some regulatory elements that are developmentally regulated may preferentially be active within certain organs or tissues at specific developmental stages, they may also be active in a developmentally regulated manner, or at a basal level in other organs or tissues within a plant as well.

By “promoter” it is meant the nucleotide sequences at the 5′ end of a coding region, or fragment thereof that contain all the signals essential for the initiation of transcription and for the regulation of the rate of transcription. There are generally two types of promoters, inducible and constitutive promoters. If tissue specific expression of the gene is desired, for example seed, or leaf specific expression, then promoters specific to these tissues may also be employed.

An inducible promoter is a promoter that is capable of directly or indirectly activating transcription of one or more DNA sequences or genes in response to an inducer. In the absence of an inducer the DNA sequences or genes will not be transcribed. Typically the protein factor that binds specifically to an inducible promoter to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus.

A constitutive promoter directs the expression of a gene throughout the various parts of an organism and/or continuously throughout development of an organism. Any suitable constitutive promoter may be used to drive the expression of the fragment of cardiac stem cell proliferation protein within a transformed cell, or all organs or tissues, or both, of a host organism. Examples of known constitutive promoters include those associated with the CaMV 35S transcript. (Odell et al., 1985, Nature, 313: 810-812). In cases where it may be desirable to produce the cardiac stem cell proliferation protein or fragment thereof in plants, plant promoters, such as, but not limited to those associated with the rice actin 1 (Zhang et al, 1991, Plant Cell, 3: 1155-1165) and triosephosphate isomerase 1 (Xu et al, 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), and the tobacco translational initiation factor 4A gene (Mandel et al, 1995 Plant Mol. Biol. 29: 995-1004) may be used.

The term “constitutive” as used herein does not necessarily indicate that a gene is expressed at the same level in all cell types, but that the gene is expressed in a wide range of cell types, although some variation in abundance is often observed.

The gene construct of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3 prime end of the mRNA precursor.

The gene construct of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified so as to increase translation of the mRNA.

The present invention further includes vectors comprising the nucleic acids as described above. Suitable expression vectors for use with the nucleic acid sequences of the present invention include, but are not limited to, plasmids, phagemids, viral particles and vectors, phage and the like. For insect cells, baculovirus expression vectors are suitable. For plant cells, viral expression vectors (such as cauliflower mosaic virus and tobacco mosaic virus) and plasmid expression vectors (such as the Ti plasmid) are suitable. The entire expression vector, or a part thereof, can be integrated into the host cell genome.

Those skilled in the art will understand that a wide variety of expression systems can be used to produce the cardiac stem cell proliferation protein or fragment thereof. With respect to the in vitro production, the precise host cell used is not critical to the invention. The cardiac stem cell proliferation protein or fragment thereof can be produced in a prokaryotic host (e.g., E. coli or B. subtilis) or in a eukaryotic host (e.g., Saccharomyces or Pichia; mammalian cells, such as COS, NIH 3T3, CHO, BHK, 293, or HeLa cells; insect cells; or plant cells). The methods of transformation or transfection and the choice of expression vector will depend on the host system selected and can be readily determined by one skilled in the art. Transformation and transfection methods are described, for example, in Ausubel et al. (1994) Current Protocols in Molecular Biology, John Wiley & Sons, New York; and various expression vectors may be chosen from those provided, e.g., in Cloning Vectors: A Laboratory Manual (Pouwels et al., 1985, Supp. 1987) and by various commercial suppliers.

In addition, a host cell may be chosen which modulates the expression of the inserted sequences, or modifies/processes the gene product in a specific, desired fashion. Such modifications (e.g., glycosylation) and processing (e.g., cleavage) of protein products may be important for the activity of the protein. Different host cells have characteristic and specific mechanisms for the post-translational processing and modification of proteins and gene products. Appropriate cell lines or host systems can be chosen by one skilled in the art to ensure the correct modification and processing of the expressed cardiac stem cell proliferation protein.

The cardiac stem cell protein or fragment thereof as defined by the present invention may be used for preventing and/or treating cardiac diseases or conditions resulting from lost, defective or damaged cardiac tissue. Without wishing to be limiting in any manner, the lost, defective or damaged cardiac tissue may be the result of a disease or disorder for example, but not limited to a heart attack, autoimmune disease, viral, bacterial or other pathogenic disease, degenerative or ischemic cardiac disease, artherosclerosis, hypertension, restenosis, angina pectoris, rheumatic heart disease, congenital cardiovascular defects, cardiac or arterial inflammation and other disease of the heart tissue/arteries, arterioles and capillaries, in the regeneration of valves, conductive tissue or vessel smooth muscle, and in the prevention of further disease in subjects undergoing coronary artery bypass graft.

Without wishing to be bound by theory or limiting in any manner, the cardiac stem cell proliferation protein or fragments thereof also may be employed to mediate gp130 signaling (stat3 activation) to exert a pro-survival effect on cardiomyocytes in vivo. As a significant component of myocardial/cardiomyocyte cell death occurs post myocardial infection (MI), the cardiac stem cell proliferation proteins or fragments thereof may limit or negate this negative effect. Further, without wishing to be bound by theory or limiting in any manner, cardiac stem cell proliferation protein or fragments thereof may promote/protect the survival of cardiac stem cells following their activation post MI using the same pathways as outlined above.

Cardiac stem cells may be isolated from a subject and contacted, directly or indirectly, with a cardiac stem cell proliferation protein or fragment thereof to promote proliferation and/or differentiation of the stem cells. Thus, the protein or fragment of cardiac stem cell proliferation protein may be useful for promoting proliferation and/or differentiation of stem cells destined for further in vitro use. Alternatively, the protein or fragment of cardiac stem cell proliferation protein may be used to stimulate the ex vivo expansion and/or differentiation of stem cells and provide a population of cells suitable for transplantation or administration to a subject in need thereof. Accordingly, the cardiac stem cells also may be administered to the same subject or to a different subject in need thereof. The protein, fragment or multimer of cardiac stem cell proliferation protein also may be used in vivo to promote proliferation and/or differentiation of resident cardiac stem cells in tissues and thereby aid in the replacement or repair of damaged tissue as a result of the disease or disorder, or after surgery or other injury.

The present invention further provides pharmaceutical compositions comprising cardiac stem cell proliferation protein or a fragment thereof and a pharmaceutically acceptable diluent, excipient or vehicle. The pharmaceutical compositions may optionally further comprise one or more stem cell modulators, one or more stem cells, or a combination thereof. Pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

Administration of the pharmaceutical compositions of the present invention may be via a number of routes depending upon whether local or systemic treatment is desired and whether a specific area is to be treated. Accordingly, the composition may be administered locally to the area to be treated. Further, the present invention contemplates parenteral administration including, but not limited to intravenous, intra-arterial, subcutaneous, intraperitoneal or intramuscular injection, for example, but not limited to intracardial injection or infusion.

As described above, the compositions of the present invention may be delivered in combination with a pharmaceutically acceptable vehicle. Preferably, such a vehicle enhances the stability and/or delivery properties. Examples may include liposomes, microparticles or microcapsules. In various embodiments of the invention, the use of such vehicles may be beneficial in achieving sustained release of the active component. The composition may also be delivered or formulated for timed-release, where the fragments of cardiac stem cell proliferation protein are released in a time-dependent manner. See WO 02/45695; U.S. Pat. No. 4,601,894; U.S. Pat. No. 4,687,757, U.S. Pat. No. 4,680,323, U.S. Pat. No. 4,994,276, U.S. Pat. No. 3,538,214, US (which are incorporated herein by reference) for several non-limiting examples of time-release formulations that may be used to assist in the time controlled release of cardiac stem cell proliferation proteins or fragments thereof within aqueous environments.

When formulated for parenteral injection, the pharmaceutical compositions are preferably used in the form of a sterile solution, containing other solutes, for example, enough saline or glucose to make the solution isotonic.

The dosage requirements for the pharmaceutical compositions vary with the particular compositions employed, the route of administration and the particular subject being treated. Typically, but not always, treatment will generally be initiated with small dosages less than the maximum or optimum dose of each compound. Thereafter the dosage is increased until the optimum effect under the circumstances is reached. In general, the pharmaceutical compositions are administered at a concentration that will generally afford effective results without causing any harmful or deleterious side effects. Administration can be either as a single unit dose, a sustained delivery dose or, if desired, the dosage can be divided into several doses that are administered at suitable times throughout the day.

When ex vivo methods of treating the stem cells are employed, the stem cells may be subsequently administered to a subject by a variety of procedures. Administration of the stem cells may be localised, and may be by injection as a cell suspension in a pharmaceutically acceptable liquid medium. Alternatively, the stem cells may be administered in a biocompatible medium which is, or becomes in situ a semi-solid or solid matrix. For example, the matrix may be an injectable liquid which forms a semi-solid gel at the site of tissue damage, such as matrices comprising collagen and/or its derivatives, polylactic acid or polyglycolic acid, or it may comprise one or more layers of a flexible, solid matrix that is implanted in its final form, such as impregnated fibrous matrices. Such matrices are known in the art (for example, Gelfoam available from Upjohn, Kalamazoo, Mich.) and function to hold the cells in place at the site of injury, which enhances the opportunity for the administered cells to proliferate and differentiate.

The present invention also contemplates administration of a nucleotide sequence encoding a cardiac stem cell proliferation protein or fragment thereof, which then expresses the encoded product in vivo. This may be accomplished via by various “gene therapy” methods known in the art. General methods of administering proteins or protein fragments are known in the art. Gene therapy includes both ex vivo and in vivo techniques. Thus, host cells may be genetically engineered ex vivo with a polynucleotide, with the engineered cells then being provided to a patient to be treated as described above.

Alternatively, cells can be engineered in vivo by administration of a cardiac stem cell proliferation protein or fragment thereof, or a nucleotide sequence encoding the same using techniques known in the art, for example, by direct injection of a “naked” polynucleotide (Feigner and Rhodes, (1991) Nature 349:351-352; U.S. Pat. No. 5,679,647) or a polynucleotide formulated in a composition with one or more other agents which facilitate uptake of the polynucleotide by the cell, such as saponins (see, for example, U.S. Pat. No. 5,739,118) or cationic polyamines (see, for example, U.S. Pat. No. 5,837,533); by microparticle bombardment (for example, through use of a “gene gun”; Biolistic, Dupont); by coating the polynucleotide with lipids, cell-surface receptors or transfecting agents; by encapsulation of the polynucleotide in liposomes, microparticles, or microcapsules; by administration of the polynucleotide linked to a peptide which is known to enter the nucleus; or by administration of the polynucleotide linked to a ligand subject to receptor-mediated endocytosis (see, for example, Wu and Wu, (1987) J. Biol. Chem. 262:4429-4432), which can be used to target cell types specifically expressing the receptors.

In another alternative, a protein/fragment-ligand complex may be formed in which the ligand comprises a fusogenic viral peptide to disrupt endosomes, allowing the cardiac stem cell proliferation protein or fragment thereof to avoid lysosomal degradation; or the protein or fragment thereof may be targeted for cell specific uptake and expression in vivo by targeting a specific receptor (see, for example, International Patent Applications WO 92/06180, WO 92/22635, WO92/20316, WO93/14188 and WO 93/20221). The present invention also contemplates the intracellular introduction of the polynucleotide and subsequent incorporation within host cell DNA for expression by homologous recombination (see, for example, Koller and Smithies (1989) Proc. Natl. Acad. Sci. USA 86:8932-8935; Zijlstra et al. (1989) Nature 342:435-438).

As described above, the polynucleotide may be incorporated into a suitable expression vector. A number of vectors suitable for gene therapy applications are known in the art (see, for example, Viral Vectors: Basic Science and Gene Therapy, Eaton Publishing Co. (2000)). In one such embodiment, which is not meant to be limiting in any manner, the expression vector may be a plasmid vector. Plasmid DNA typically does not integrate into the genome of the host cell, but is maintained in an episomal location as a discrete entity eliminating genotoxicity issues that chromosomal integration may raise. A variety of plasmids are available commercially and include those derived from Escherichia coli and Bacillus subtilis, with many being designed particularly for use in mammalian systems. Examples of plasmids that may be used in the present invention include, but are not limited to, the eukaryotic expression vectors pRc/CMV (Invitrogen), pCR2.1 (Invitrogen), pAd/CMV and pAd/TR5/GFPq (Massie et al., (1998) Cytotechnology 28:53-64). In an exemplary embodiment, the plasmid is pRc/CMV, pRc/CMV2 (Invitrogen), pAdCMV5 (IRB-NRC), pcDNA3 (Invitrogen), pAdMLP5 (IRB-NRC), or pVAX (Invitrogen).

Alternatively, the expression vector may be a viral-based vector. Examples of viral-based vectors include, but are not limited to, those derived from replication deficient retrovirus, lentivirus, adenovirus and adeno-associated virus. Retrovirus vectors and adeno-associated virus vectors are currently the recombinant gene delivery system of choice for the transfer of exogenous genes in vivo, particularly into humans. These vectors provide efficient delivery of genes into cells, and the transferred polynucleotides are stably integrated into the chromosomal DNA of the host. A major prerequisite for the use of retroviruses is to ensure the safety of their use, particularly with regard to the possibility of the spread of wild-type virus in the cell population. Retroviruses, from which retroviral vectors may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumour virus. Specific retroviruses include pLJ, pZIP, pWE and pEM, which are well known to those skilled in the art.

The polynucleotide may be incorporated into the vector under the control of a suitable promoter that allows for expression of the encoded polypeptide in vivo. Suitable promoters which may be employed include, but are not limited to, adenoviral promoters, such as the adenoviral major late promoter, the E1A promoter, the major late promoter (MLP) and associated leader sequences or the E3 promoter; the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTR; the histone, pol III, and (α-actin promoters; B19 parvovirus promoter; the SV40 promoter; and human growth hormone promoters). The promoter also may be the native promoter for the gene of interest. The selection of a suitable promoter will be dependent on the vector, the host cell and the encoded protein and is considered to be within the ordinary skills of a worker in the art.

The development of specialised cell lines (also referred to as “packaging cells”) that produce only replication-defective retroviruses has increased the utility of retroviruses for gene therapy, and defective retroviruses are well characterised for use in gene transfer for gene therapy purposes (for a review see Miller, A. D. (1990) Blood 76:271). Thus, recombinant retrovirus can be constructed in which part of the retroviral coding sequence (gag, pol, env) has been replaced by subject polynucleotide and renders the retrovirus replication defective. The replication defective retrovirus is then packaged into virions that can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.), J. Wiley & Sons, (1989), Sections 9.10-9.14 and other standard laboratory manuals. Examples of suitable packaging virus lines for preparing both ecotropic and amphotropic retroviral systems include Crip, Cre, 2 and Am. Other examples of packaging cells include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy, Vol. 1, pgs. 5-14 (1990).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Neda et al. (1991) J Biol Chem 266:14143-14146). Coupling can be in the form of the chemical cross-linking with a protein or other variety (for example, lactose to convert the env protein to an asialoglycoprotein), as well as by generating fusion proteins ((for example, single-chain antibody/env fusion proteins). This technique, while useful to limit or otherwise direct the infection to certain tissue types, may also be used to convert an ecotropic vector in to an amphotropic vector.

Moreover, use of retroviral gene delivery can be further enhanced by the use of tissue- or cell-specific transcriptional regulatory sequences which control expression of the polynucleotides contained in the vector.

Another viral vector useful in gene therapy techniques is an adenovirus-derived vector. The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (for example, Ad2, Ad3, Adz etc.) are well known to those skilled in the art. Recombinant adenoviruses can be advantageous in certain circumstances in that they can be used to infect a wide variety of cell types. Furthermore, the virus particle is relatively stable and amenable to purification and concentration, and as above, can be modified so as to affect the spectrum of infectivity. Additionally, introduced adenoviral DNA (and foreign DNA contained therein) is not always integrated into the genome of a host cell but can remain episomal, thereby avoiding potential problems that can occur as a result of insertional mutagenesis in situations where introduced DNA becomes integrated into the host genome (for example, retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and contemplated by the present invention are deleted for all or parts of the viral E2 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127).

Generation and propagation of replication-defective human adenovirus vectors usually requires a unique helper cell line. Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus, i.e. that provide, in trans, a sequence necessary to allow for replication of a replication-deficient virus. Such cells include, for example, 293 cells, Vero cells or other monkey embryonic mesenchymal or epithelial cells. The use of non-human adenovirus vectors, such as porcine or bovine adenovirus vectors is also contemplated. Selection of an appropriate viral vector and helper cell line is within the ordinary skills of a worker in the art.

In one embodiment of the present invention, the gene therapy vector is an adenovirus-derived vector.

The present invention additionally provides for therapeutic kits containing one or more cardiac stem cell proliferation proteins or fragments thereof and optionally one or more stem cell modulators, alone or in combination with one or more pharmaceutical compositions, diluents or the like. Individual components of the kit may be packaged in separate containers if desired. The kits also may comprise instructions on how to use one or more of the components therein.

When the components of the kit are provided in one or more liquid solutions, the liquid solution can be an aqueous solution, for example a sterile aqueous solution. In this case the kit may further comprise a syringe, pipette, eye dropper, catheter, loadable osmotic pump or other such apparatus, from which the protein, fragment or multimer or composition comprising the same may be administered and/or delivered to a patient or subject.

The components of the kit may also be provided in dried or lyophilised form and the kit can additionally contain a suitable solvent for reconstitution of the dry or lyophilised components. Irrespective of the number or type of containers, the kits of the invention also may comprise an instrument for assisting with the administration of the composition to a patient.

The present invention also contemplates methods of purifying cardiac stem cell proliferation protein or fragments thereof from tissue culture medium. For example, but not to be considered limiting in any manner, a method was developed by which cardiac stem cell proliferation protein, fragments or multimers thereof can be produced in high yield using the well known GST-fusion bacterial expression system coupled with chromatographic-based affinity purification schemes. More recently, alternate approaches for producing functional cardiac stem cell proliferation protein or fragments thereof from either murine or human sources were explored as described below.

The human cardiac stem cell proliferation protein cDNA (or fragment thereof) containing a 5′ nerve growth sequence (NGS) is inserted into the pShuttle viral expression vector (Stratagene). This vector uses the CMV promoter for high level expression of the gene product. Cells infected with adenoviral cardiac stem cell proliferation protein or a fragment thereof produce the product and subsequently secrete it into the tissue culture medium via the NGS. The tissue culture media can be collected, concentrated and then methods in ion exchange and size exclusion chromatography may be employed to purify cardiac stem cell proliferation protein. Such a strategy can be adapted to produce any cardiac stem cell proliferation protein or fragment thereof intended for clinical evaluation.

The invention will now be described in detail by way of reference only to the following non-limiting examples.

### Example 1

### Cardiac Stem Cell Proliferation Protein and Fragments Thereof

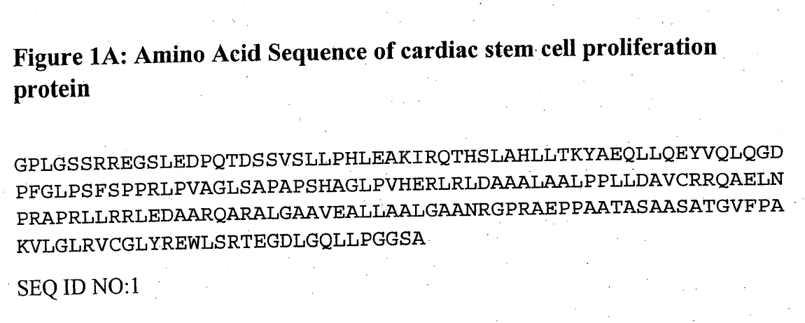

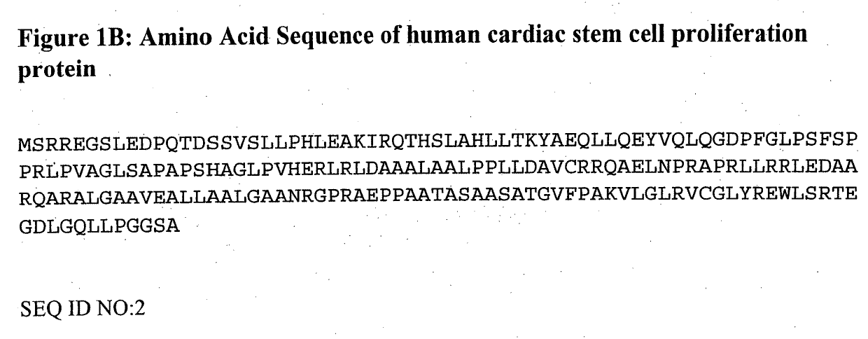

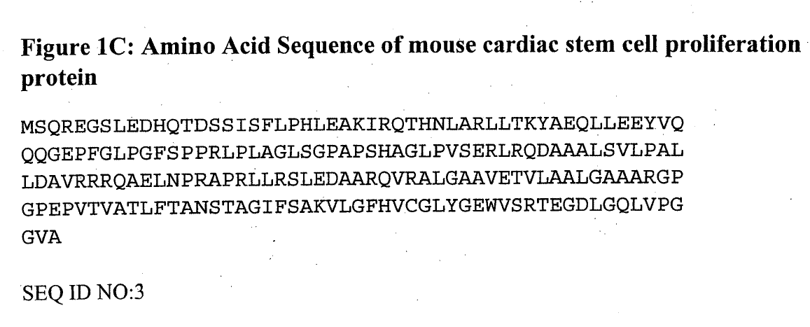

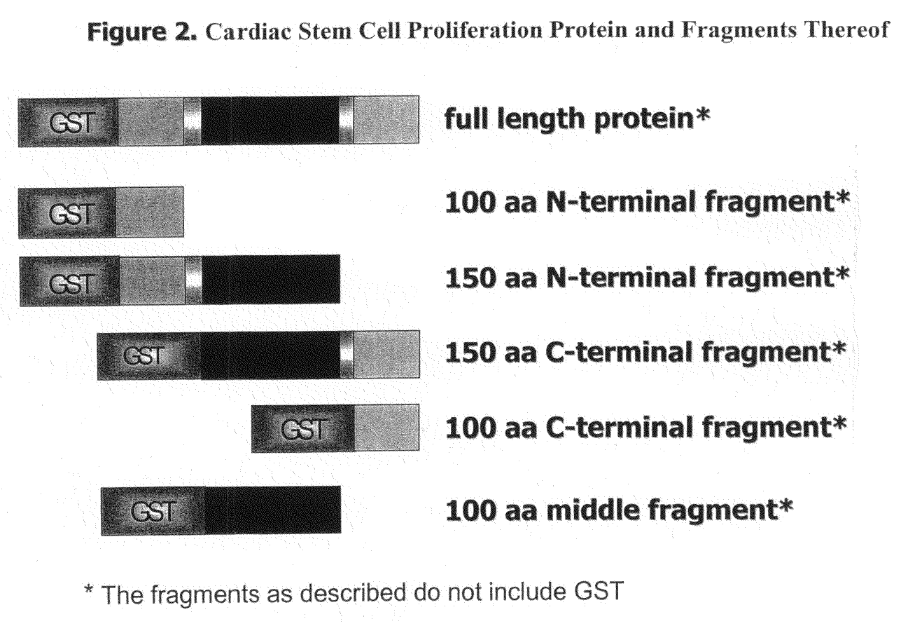

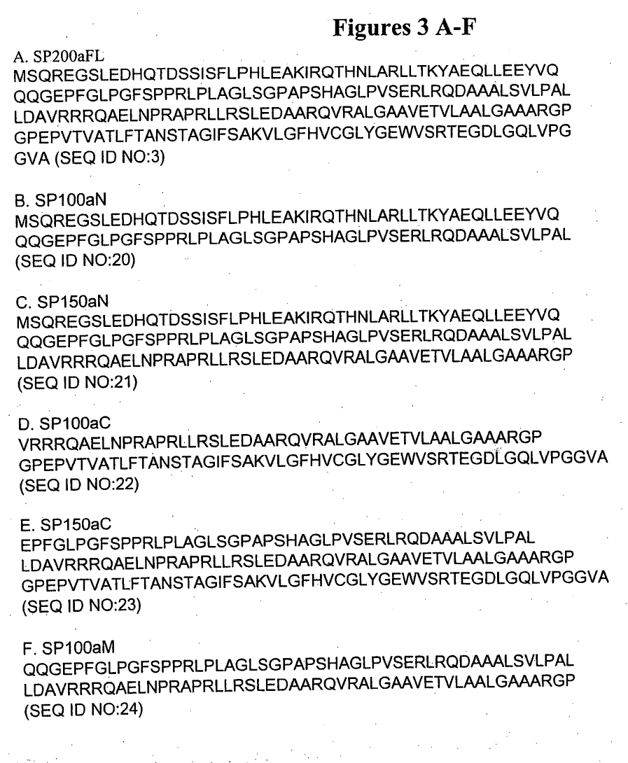

The fragments of cardiac stem cell proliferation protein chosen to exemplify the present invention, but without wishing to be limiting in any manner were based on the mouse sequence (FIG. 1C; SEQ ID NO:3). The full length murine protein is 203 amino acids in length (SEQ ID NO:3). 6 fragments were prepared: a 100 amino acid fragment from the N-terminus (SEQ ID NO:20), a 150 amino acid fragment from the N-terminus (SEQ ID NO:21), a 100 amino acid fragment from the C-terminus (SEQ ID NO:22), a 150 amino acid fragment from the C-terminus (SEQ ID NO:23) and a 100 amino acid fragment from the middle of the full length proteins (SEQ ID NO:24) (see FIG. 2). The sequences of each fragment are shown in FIGS. 3 A-F.

A glutathione S-transferase (GST) affinity tag was placed at the N-terminus of each fragment to facilitate purification. The tags could be removed to produce the desired fragment of cardiac stem cell proliferation protein. The size of the fragments listed in FIG. 2 does not include the GST affinity tag.

### Example 2

### Gene Constructs and Assembly of Vectors Expressing Fragments of Cardiac Stem Cell Proliferation Protein

Gene constructs for each of the six different GST-fragments described in Example 1 were prepared using known recombinant DNA techniques (Sambrook et al (1989) Molecular cloning: A laboratory Manual, Sec Ed. Cold Spring Harbor Laboratory press, Cold Spring Harbor, N.Y.). Table 1 provides a description of the constructs names as used herein for the nucleotide sequences encoding fragments of cardiac stem cell proliferation protein.

TABLE 1
DNA constructs for cardiac stem cell proliferation protein
fragments

| Construct/Name | Description |
|---|---|
| SP200aFL | Full-length cardiac stem cell proliferation protein, |
| (SEQ ID NO: 3) | 203 amino acids, not including GST tag |
| SP100aN | N-terminal 100 amino acids, not including GST tag |
| (SEQ ID NO: 20) | |
| SP150aN | N-terminal 150 amino acids, not including GST tag |
| (SEQ ID NO: 21) | |
| SP100aC | C-terminal 103 amino acids, not including GST tag |
| (SEQ ID NO: 22) | |
| SP150aC | C-terminal 153 amino acids, not including GST tag |
| (SEQ ID NO: 23) | |
| SP100aM | Middle 100 amino acids (amino acids 61-160), not |
| (SEQ ID NO: 24) | including GST tag |

The full length murine cardiac stem cell proliferation protein cDNA sequence was PCR amplified and cloned into the pGEX6.p1 expression vector (Amersham) containing an N-terminal sequence for the glutathione S-transferase tag. PCR amplification of the murine cardiac stem cell proliferation protein cDNA was performed to also generate the N- and C-terminal fragments as described above. Primers were determined based on the DNA sequences that when translated, would yield 100 amino acids from the N- or C-terminus, 150 amino acids from the N- or C-terminus, and a 100 amino acid fragment located within the cardiac stem cell proliferation protein core (middle fragment). As will be evident to a person of skill in the art, similar or other procedures may be employed to create fragments of cardiac stem cell proliferation protein other than those listed above.

The plasmids containing each construct were sequence verified, then transformed into the E. coli strain BL21 (DE3)RIL cells (Promega). Transformation was performed in BL21 (DE3) cells modified to compensate for E. coli codon bias (Promega BL21 CodonPlus Instruction Manual revision # 0805008 and references within). IPTG-induced expression of each transformed plasmid was performed and the protein product was tested for proper yield and size using SDS-PAGE and Coomassie blue stain.

### Example 3

### Expression and Purification of Cardiac Stem Cell Proliferation Protein Fragments

To obtain large quantities of purified recombinant cardiac stem cell proliferation protein and fragments thereof, large scale protein expression was performed.

The BL21 (DE3)RIL strain carrying the cardiac stem cell proliferation protein construct of interest was grown overnight in a 200 ml LB+Ampicillin starter culture at 37° C. The starter culture was then diluted 1:10 using 2×YT (Bacto-tryptone 16 g, Bacto-Yeast extract 10 g, NaCl 5 g, ddH₂O up to 1 L). The culture was placed at 37° C. in a bacterial shaker incubator for approximately 1.5-2 hrs. When the culture was in log phase (Absorbance at 600 nm is 0.6-1.0), protein expression as induced using IPTG to a final concentration of 0.1 mM. Incubation at 37° C. in the bacterial shaker incubator continued for approximately 4-5 hrs. The culture was then harvested by centrifuging at 6000×g for 15 min. at 4° C., the supernatant was discarded and the bacterial pellet was frozen at −20° C.

The bacterial pellet was thawed on ice for 15 min., then resuspended in 10 ml STE Buffer (10 ml 1M Tris Base (pH 8.0), 5M NaCl 30 ml, 0.5M EDTA (pH8.0) 2 ml, ddH₂O up to 1 L). 100 μl of 10 mg/ml lysozyme solution was added and the mixture was placed on ice for 20 min. 100 μL of 1M DTT and 700 μL of 10% Sarcosine (made in STE Buffer) were added and the mixture was transferred to a 50 ml conical tube. The mixture was sonicated on ice for 2 min. with 2 second on/off pulses, then centrifuged at 16,000 rpm for 20 min. at 4° C. The supernatant was decanted, then supplemented with 4 ml 10% Triton™ X-100 (in STE Buffer) and topped up to 20 ml with STE Buffer. The bacterial lysate was left at room temperature for 30 min, then stored at 4° C.

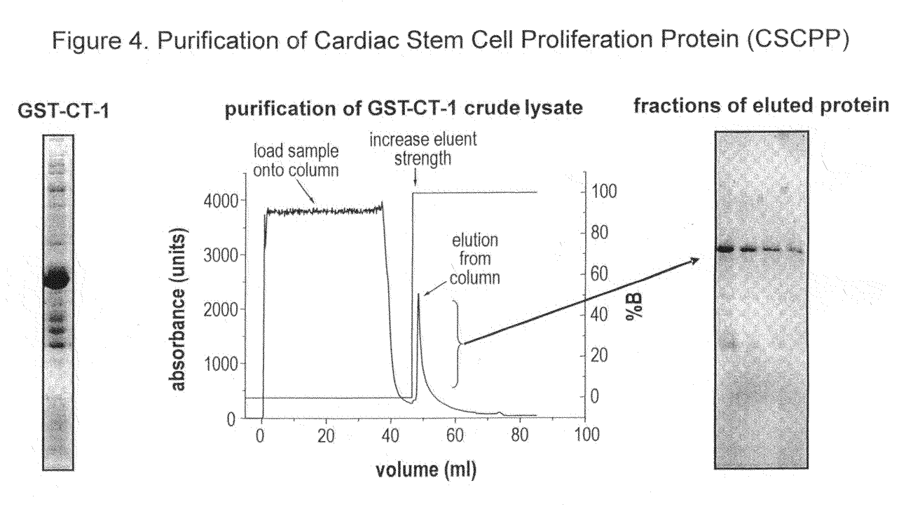

Purification of the bacterial lysate was carried out with a fast protein liquid chromatography (FPLC) platform on the AKTA10 FPLC (Amersham) using GST affinity columns (GSTrap FF). The GSTrap FF column was equilibrated with 10 column volumes (CV) of PBS at a flow rate of 4.0 ml/min. Approximately 40 ml of prepared bacterial lysate was loaded onto the sample loop and was applied onto the column at a flow rate of 0.25 ml/min. The column was then washed with 40 CV of PBS at 4.0 ml/min. The bound GST-protein was eluted from the GSTtrap column using 3 CV of 10 mM glutathione (pH 8.6) and collected in 1.0 ml fractions (FIG. 4). In some instances, the bound sample, once washed of non-specific proteins was incubated overnight at 4 deg with PreScission protease as described below and then eluted from the column by washing with 10 CV of PBS. The fractions of interest, determined by the absorbance at OD₂₈₀ were pooled and desalted in PreScission buffer (50 mM Tris pH 7.0, 150 mM NaCl, 1 mM EDTA) using the AKTA10 FPLC and a 26/10 desalting column (Amersham). The desalted protein was collected in 1.0 ml fractions and pooled. The sample was concentrated using 1K MicroSep centrifugal concentrators to an approximate volume of 1.0 ml. The protein concentration was determined by Bradford assay (BioRad).

The sample was treated to remove the GST-tag. Specifically, the protein solution was incubated with PreScission buffer containing 1 mM DTT and the PreScission Protease (Amersham) at a concentration of 1 μl protease/100 μg protein. This reaction was left overnight at 4° C. The GST-tag and the PreScission protease were removed from the final protein solution by purification through a GSTrap FF column. The concentration of the final protein solution was determined using the Bradford assay, then stored at −20° C. This purification protocol was used for all GST-cardiac stem cell proliferation protein constructs. However, in parallel experiments, GST-cardiac stem cell proliferation protein fragment fusion proteins were tested for biological activity.

### Example 4

### In Vitro Assay of Cardiac Stem Cell Proliferation Protein Activity

The activity of full length murine cardiac stem cell proliferation protein (SP200aFL) was assayed in h9c2 cells (rat cardiomyocytes) and the activity of full length human cardiac stem cell proliferation protein (hSP200aFL) was assayed in LLCPK1A cells (pig epithelial). The activity of the 100 carboxy-terminal amino acids of murine cardiac stem cell proliferation protein (SP100aC) was also assayed in h9c2 cells. The assays used were based on the activation of a signaling pathway which is known to result in STAT3 tyrosine phosphorylation.

The rat myocardial h9c2 cells (ATCC CRL-1446) were grown in Dubelco modified eagle medium (DMEM) supplemented with 10% fetal bovine serum (FBS) and 1× penicillin/streptomycin. Cells were maintained at 37° C. with 5% CO₂. The pig (Sus scrofa) epithelial LLCPK1A cells (ATCC CL-101.1) were maintained in Media 199 containing 5% FBS supplemented with 1× penicillin/streptomycin, and incubated at 37° C.

H9c2 cells were grown to approximately 80% confluency, then serum starved (DMEM without FBS and penicillin/streptomycin) for approximately 4 h. The cells were treated with 1.0 μg of purified recombinant SP200aFL in DMEM and harvested after 15, 30, 60, 120, 240 and 360 minutes. A separate group of cells were either left in DMEM without treatment (−) or exposed to DMEM with FBS (+) for 360 min.

Cells were harvested and protein lysates were prepared by collecting tissue culture cells in ice cold PBS, then homogenizing in modified radio immune precipitation assay (RIPA). Modified RIPA (150 mM NaCl, 50 mM Tris pH 7.5, 1 mM EDTA, 1% NP40, 1% Triton™ X-100) was supplemented with 10 mM pepstatin, 10 mM aprotinin, 10 mM leupeptin, 50 mM PMSF, 20 mM NaF, and 10 mM Na₃VO₄. Cell lysates were centrifuged at 12,000×g for 30 min, aliquoted and stored at −80° C.

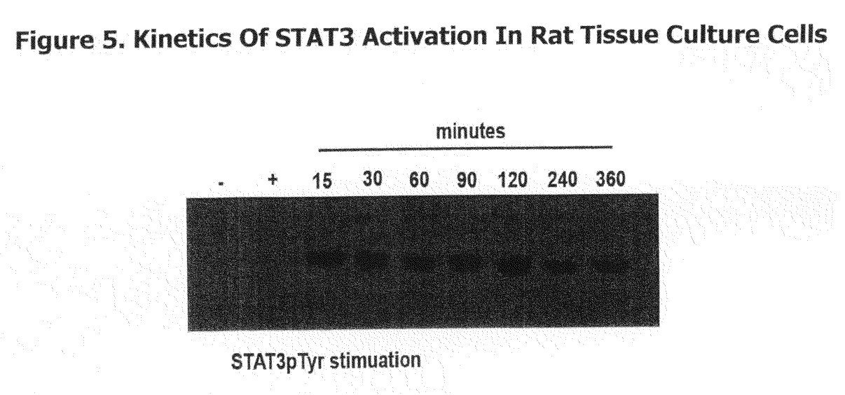

Approximately 50 μg of total protein was electrophoresed through an 8% SDS-polyacrylamide gel and Western blotted, as described above. Results (FIG. 5) show that phospho-tyrosine STAT3 (STAT3pTyr) was elevated after 15 and 30 minutes of stimulation with the full length murine cardiac stem cell proliferation protein (SP200aFL; SEQ ID NO:3), with a transient increase noted after 120 minutes. Activity of STAT3pTyr was still detected after 360 min of initial stimulation with SP200aFL. The results also demonstrate that murine stem cell proliferation protein can activate the STAT-3 signalling pathway in rat cells.

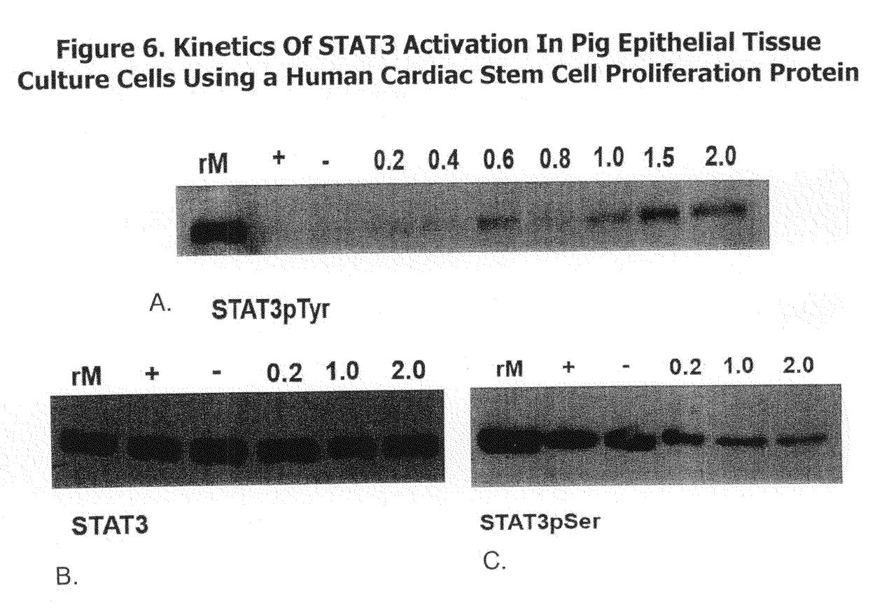

LLPCK1A cells were grown to approximately 80% confluency, then serum starved (M199 without FBS) for 6 h. The full length human cardiac stem cell proliferation protein (hSP200aFL; SEQ ID NO:2) was added in varying concentrations (μg protein) to LLPCK1A cells for 30 min. A separate group of cells were treated with recombinant mouse cardiac stem cell proliferation protein (rM), received serum starvation only (−), or were treated with M199+FBS (+). Cells were harvested and protein lysates prepared as described above. Results shown at FIG. 6A indicate that STAT3pTyr levels increased with concentrations of hSP200aFL of up to about 0.6 μg, then increased again at about 1.5 μg of hSP200aFL (SEQ ID NO:2). Without wishing to be bound by theory or limiting in any manner, the results suggest that about 1.5 μg of hSP200aFL stimulates STAT3 tyrosine phosphorylation and that higher concentrations potentially may inhibit phosphorylation. A subsequent analysis (FIG. 6B) demonstrated that both low and high concentrations of hSP200aFL had similar effects on STAT3 serine phosphorylation (STAT3pSer). In addition, the levels of total STAT3 were similar under the tested conditions (FIG. 6C). These results also demonstrate that human cardiac stem cell proliferation protein can activate the STAT-3 signalling pathway in porcine cells.

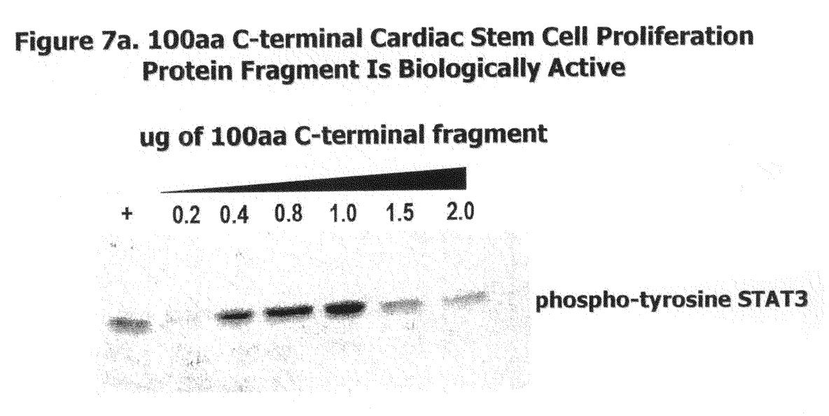

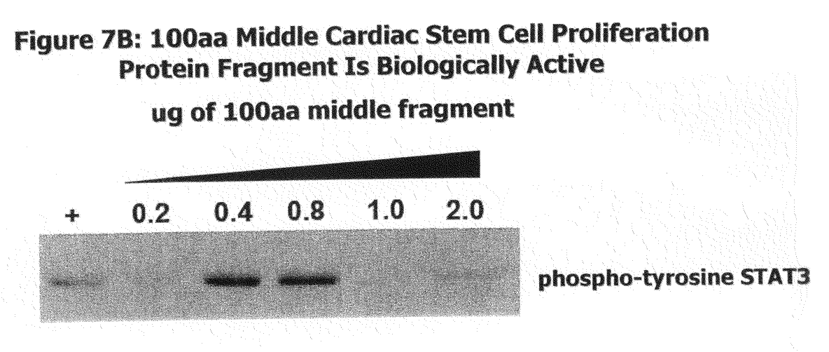

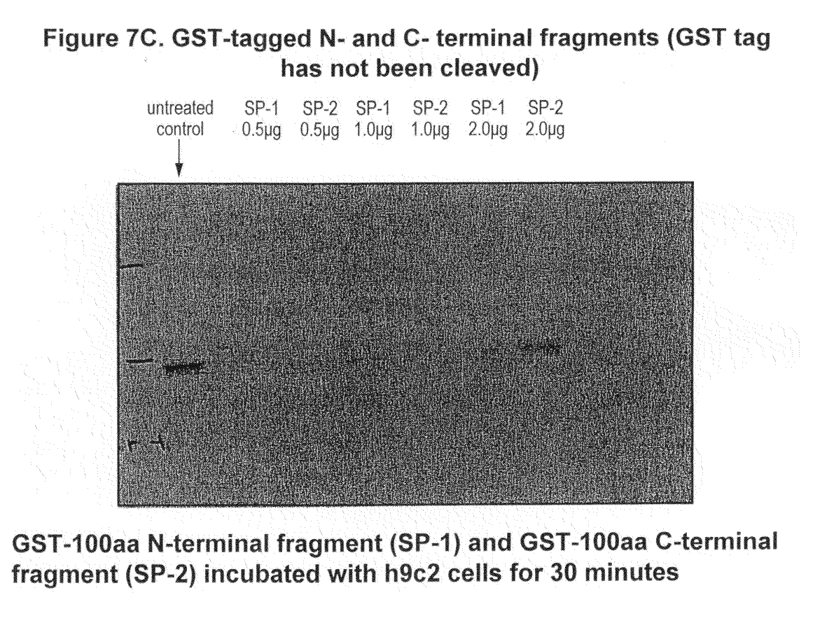

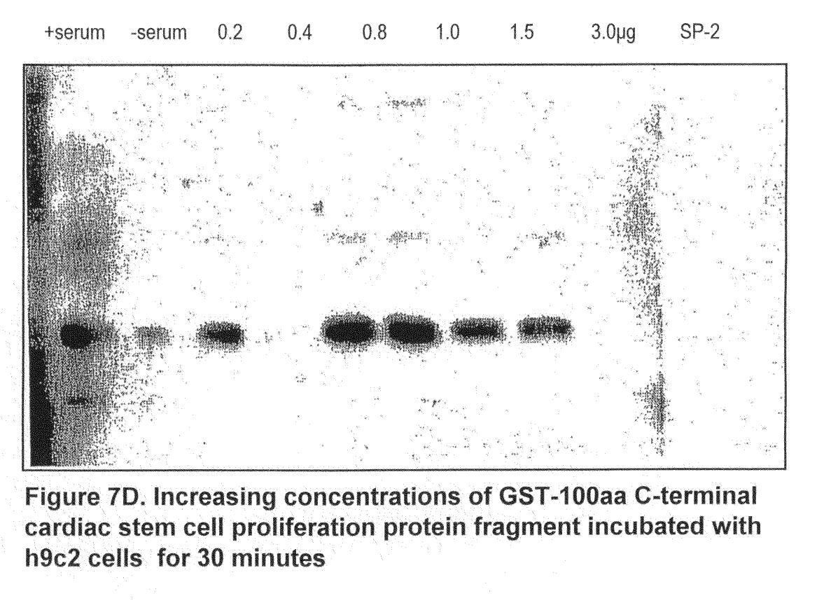

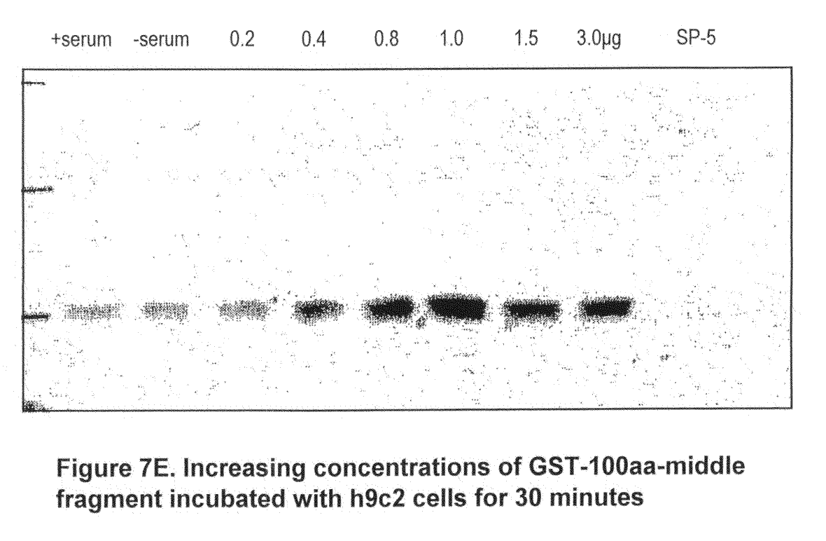

The one-hundred amino acid carboxyl-terminus fragment of cardiac stem cell proliferation protein (SP100aC; see FIG. 3), or the one hundred amino acid middle fragment of cardiac stem cell proliferation protein (SP100aM) was expressed and purified as described in Examples 2-3, above. h9c2 cells were incubated with varying concentrations of SP100aC or SP100aM for 30 minutes, harvested and analyzed for STAT3 activity as described above. As shown in FIGS. 7a,b SP100aC and SP100aM stimulated tyrosine phosphorylation of STAT3 in a dose dependent manner. Further, as shown in FIGS. 7c-e, the 100 amino acid N-terminal, C-terminal and middle fragment were capable of stimulating STAT3 phosphorylation even when the constructs also comprised an N-terminal GST amino acid sequence. These results suggest that fragments of cardiac stem cell proliferation protein are capable of activating the STAT3pTyr pathway and importantly that fragments of murine cardiac stem cell proliferation protein activate the STAT3pTyr pathway in rat cells.

### Example 5

### In Vivo Assay of Cardiac Stem Cell Proliferation Protein Activity in Rats

The effect of recombinant cardiac stem cell proliferation protein on STAT3 signaling in rats was determined.

Alzet™ mini osmotic pumps were loaded with recombinant murine SP200aFL (25 ng/g body weight) under sterile conditions and implanted subcutaneously under the dorsal skin of Sprague-Dawley rats. The pumps were set to release approximately 611/day of SP200aFL for 30 days, which amounted to about 150 ng of protein/day/g body weight. The rats were sacrificed at various time points as indicated (days) and heart tissue was collected. Tissues were harvested and flash frozen in liquid nitrogen. Frozen tissues were powdered under liquid nitrogen and homogenized in modified RIPA buffer and supplements, as described in Example 4, using a dounce homogenizer. Lysates were centrifuged for 45 minutes and the supernatant was aliquoted and stored at −80° C.

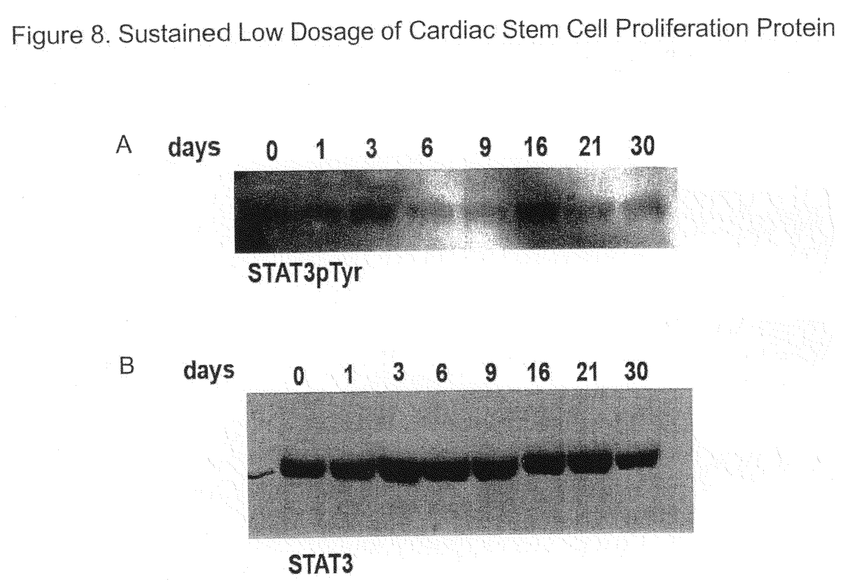

FIG. 8A shows the Western analyses of heart tissue lysates, which indicated that administration of cardiac stem cell proliferation protein resulted in STAT3 phosphorylation. Further, the results suggest that STAT3pTyr was elevated after 3 and 16 days. In addition, the levels of total STAT3 were not changed (FIG. 8B). This data suggests that cardiac stem cell proliferation protein or fragments thereof can be administered to a subject to induce signalling pathways, as observed by STAT3 phosphorylation in the heart.

Accordingly, the present invention provides a method of delivering cardiac stem cell proliferation protein or a fragment thereof to a subject, the method comprising the step of administering a cardiac stem cell proliferation protein or fragment thereof to the subject. In a preferred embodiment, the fragment is administered in a single continuous low dose rather than in a single dose or multiple doses. Preferably, the low single continuous dose is administered over the course of one or several days, for example but not limited to from about 1 to about 30 days. Without wishing to be limiting in any manner, such a dose may be delivered by pump, for example, but not limited to an osmotic pump. Other methods of continuously delivering cardiac stem cell proliferation protein, or a fragment thereof, as would be known in the art are also encompassed by the methods of the present invention.

### Example 6

### In Vivo Assay of Cardiac Stem Cell Proliferation Protein Activity in Pigs

The effect of recombinant adenoviral cardiac stem cell proliferation protein on STAT3 signaling in pigs was determined.

Adenoviral constructs expressing the human cardiac stem cell proliferation protein nucleotide sequence were tested in pigs. The human cardiac stem cell proliferation protein cDNA sequence with a 5′ RSV promoter and a human nerve growth factor (NGF) signal sequence was inserted into the pShuttle vector of the AdEasy adenoviral vector system (Stratagene) as described by the manufacturer (Stratagene catalogue #240009, manual revision #066004j). The adenovirus was generated according to instructions listed in Stratagene catalogue #240009, manual revision #066004j.

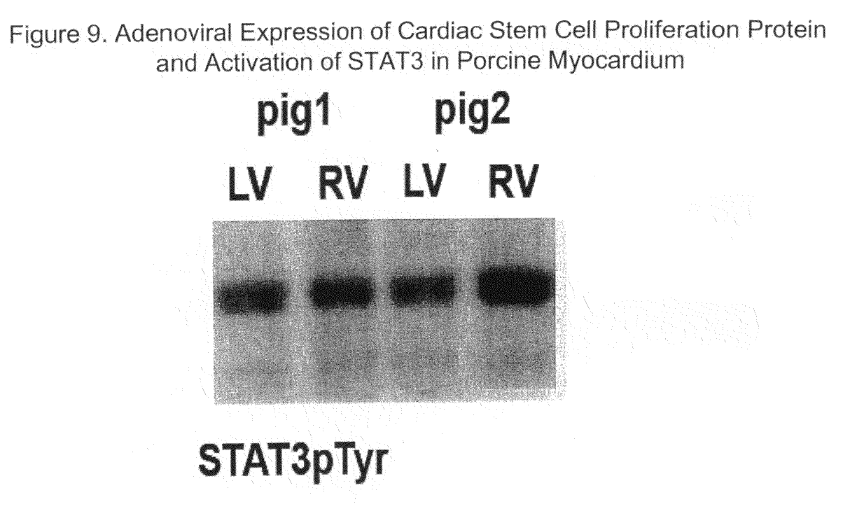

Sedentary pigs (pig1 and pig20) were injected with adenoviral cardiac stem cell proliferation protein and monitored for 14 days. The left and right ventricle (LV and RV, respectively) were analyzed for phosphotyrosine STAT3. STAT3pTyr was detected in both the left and right ventricle using an anti-STAT3 phosphotyrosine antibody (FIG. 9). These results demonstrate that STAT3 activity can be readily detected in porcine hearts after administration of an adenoviral vector comprising cardiac stem cell proliferation protein or a fragment thereof.

Accordingly, the present invention contemplates a method of delivering cardiac stem cell proliferation protein or a fragment thereof to a subject as defined above, wherein the cardiac stem cell proliferation protein or fragment thereof is encoded and expressed nucleotide sequence from an adenoviral vector.

### Example 7

### In Vivo Assay of Cardiac Stem Cell Proliferation Protein Fragment Activity in Rats and Pigs

The study involved a total of 3 pigs. Pigs were anaesthetized orally with metoprolol prior to inducing a myocardial infarction using a 7 Fr. Sheath placed in right femoral artery via the modified Seldinger technique (Seldinger SI Catheter replacement of the needle in percutaneous arteriography: a new technique. Acta Radiol. 1953; 39:368-376). Two of these animals received adenoviral cardiac stem cell proliferation protein immediately after infarct and the third received adenovirus expressing cardiac stem cell proliferation protein 48 h after the infarct. The adenovirus was resuspended in sterile saline to a concentration of 3.0×10⁸ PFU/ml in a total volume of 100 ml. The adenovirus solution was infused via the ear vein for a duration of 45 min. The pigs were re-anaesthetized 48 hrs post-infarct and their cardiac tissue imaged by magnetic resonance imaging (MRI) for left ventricular function, volume, mass and infarct size. Each pig received a second dose of adenoviral cardiac stem cell proliferation protein after 14 days in the same manner as the first dose (ear vein infusion of 3.0×10⁸ PFU/ml adenovirus cardiac stem cell proliferation protein in 100 ml sterile saline for 45 min). A final MRI scan was performed at 5-6 weeks post-MI and the pigs were sacrificed

In a preferred embodiment, the cardiac stem cell proliferation protein or fragment thereof is delivered at a dose less than about 25 μg/kg weight. More preferably, the cardiac stem cell proliferation protein or fragment thereof is delivered in an amount between about 12.5 μg/kg and about 0.01 μg/kg, for example but not limited to 12.5, 10, 7.5, 6.25, 5, 2.5, 1, 0.75, 0.5, 0.25, 0.1, 0.09, 0.08, 0.07, 0.06, 0.05, 0.04, 0.03, 0.02, 0.01 μg/kg, or any amount therein between. Similarly, the amount may be defined by a range of any two of the values listed above or any two values therein between. A preliminary safety study was conducted wherein cardiac stem cell proliferation protein was administered to two pigs at 12.5 μg/kg and 6.25 μg/kg respectively without inducing an infarct. Both pigs survived and remained healthy.

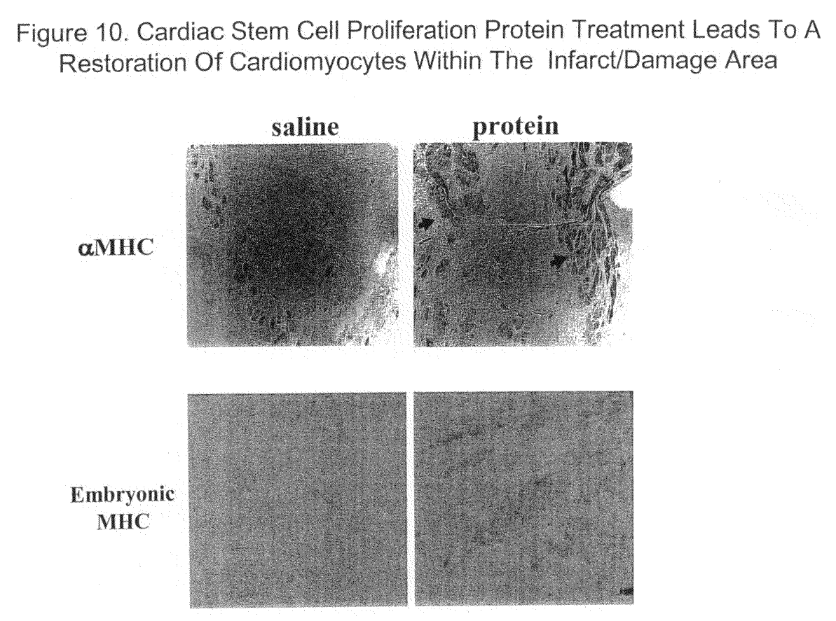

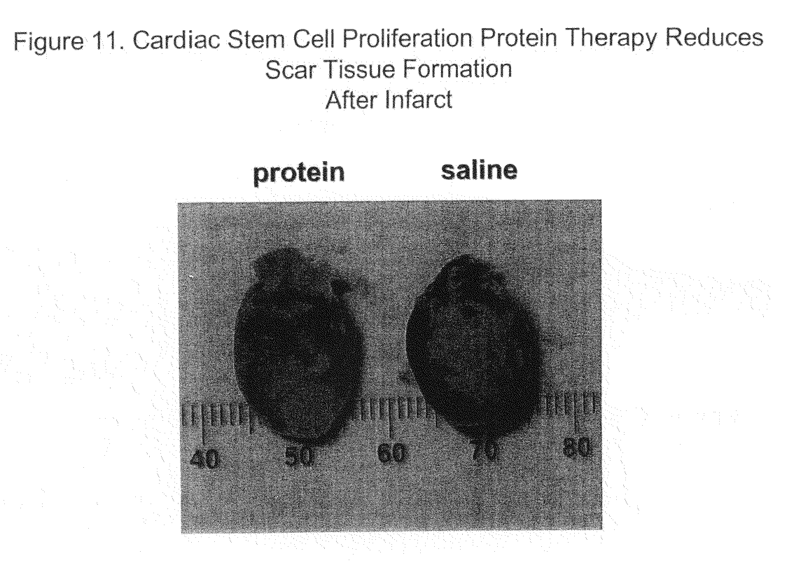

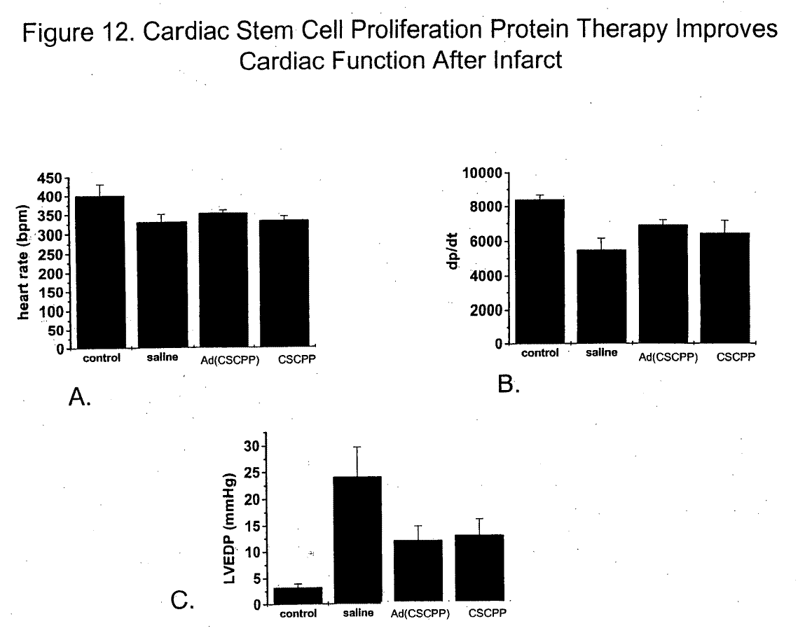

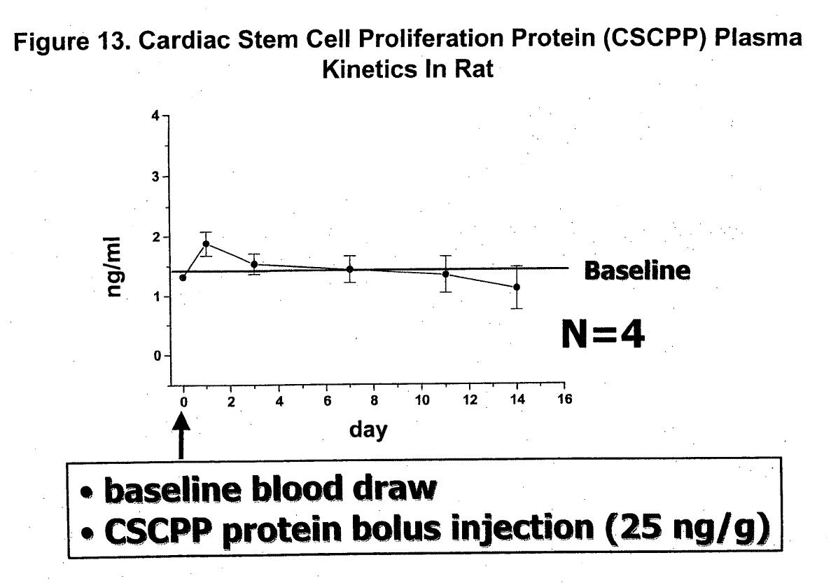

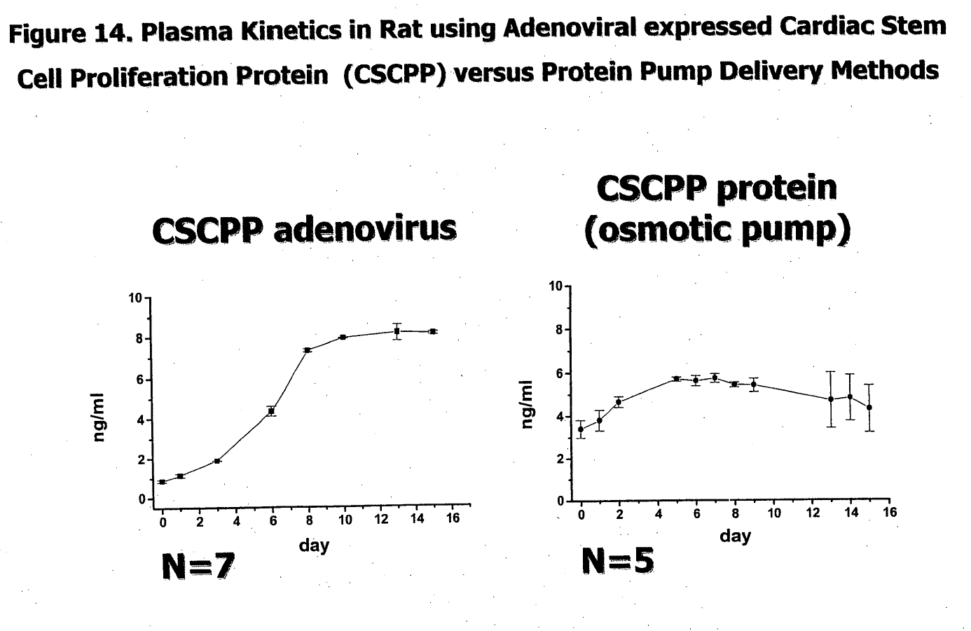

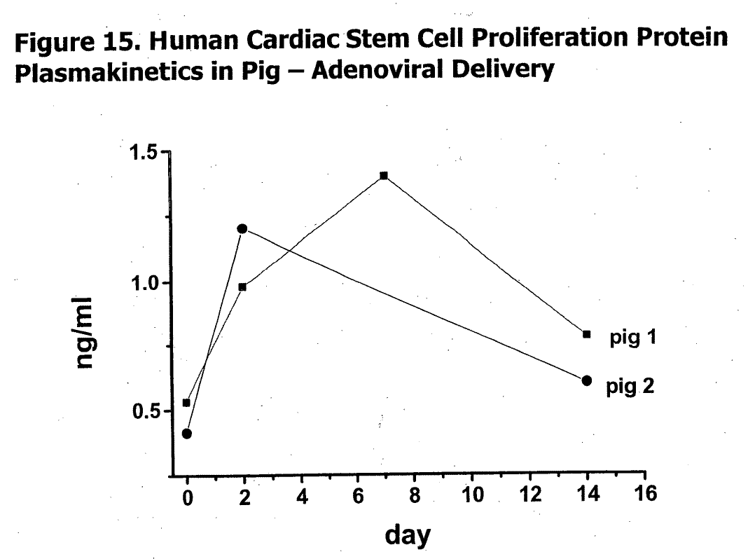

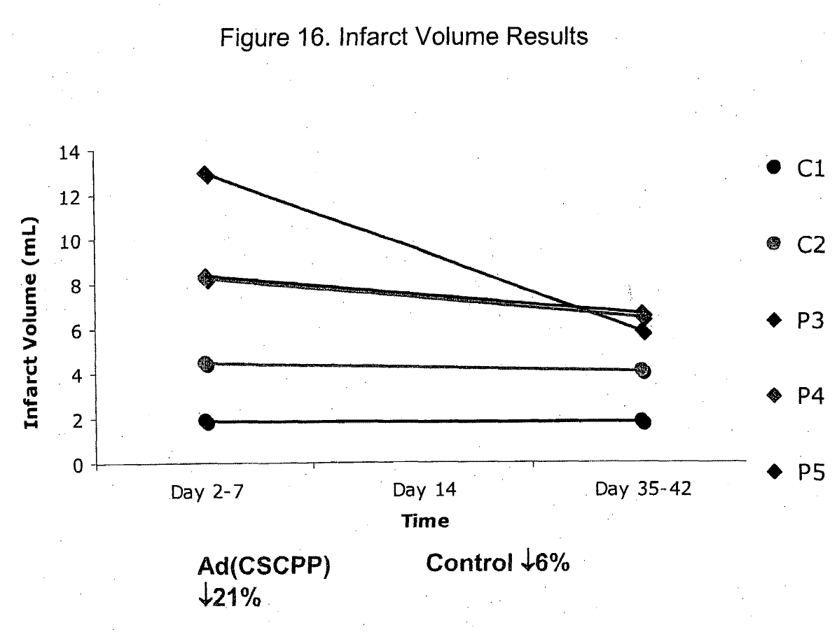

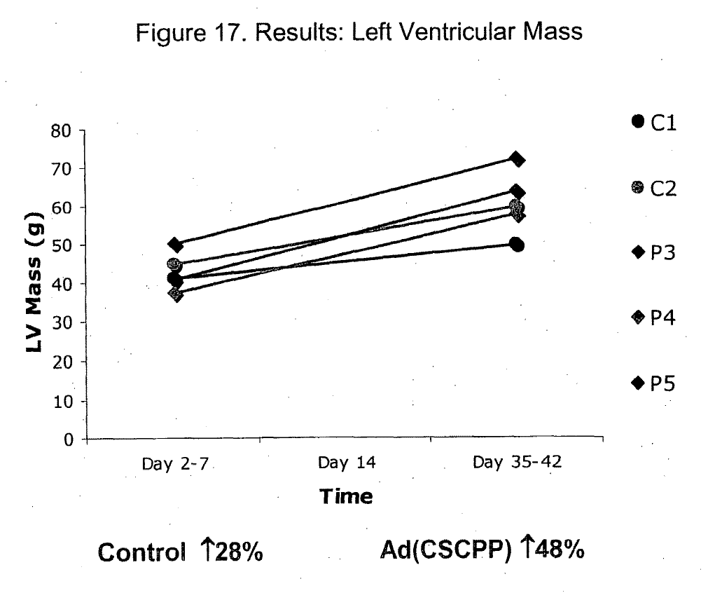

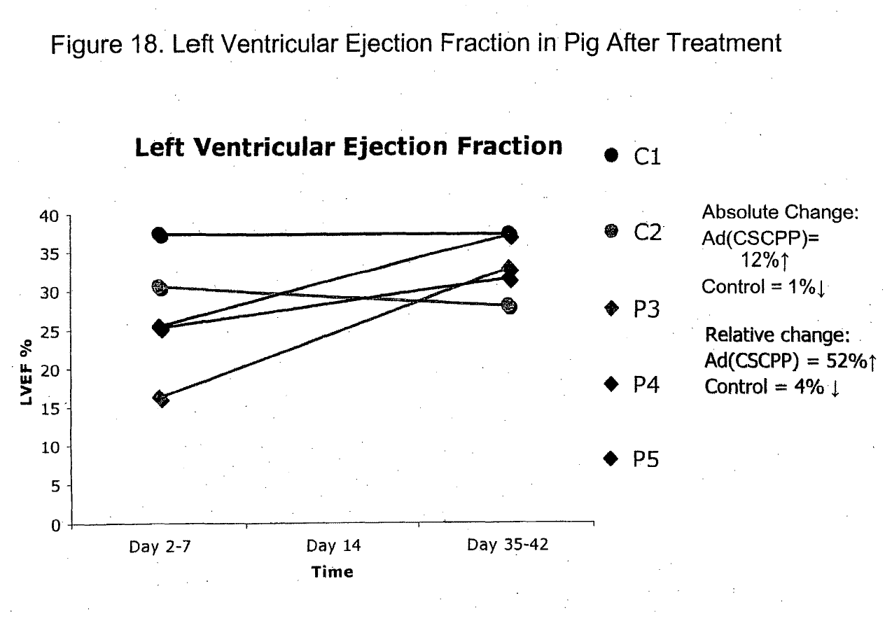

The results of the infarct experiments described above suggest that cardiac stem cell proliferation protein treatment restores cardiomyocytes within the infarct (FIG. 10), reduces scar tissue formation (FIG. 11), and improves cardiac function (FIG. 12). In respect of rats administered cardiac stem cell proliferation protein or adenovirus capable of expressing cardiac stem cell proliferation protein, as shown in FIG. 13, administration of a single cardiac stem cell proliferation protein bolus injection in an amount of about 25 ng/g resulted in a plasma concentration of about 1 to about 2 ng/ml of cardiac stem cell proliferation protein over a period of about 14 days. As shown in FIG. 14a adenoviral-produced cardiac stem cell proliferation protein showed a biphasic response wherein plasma cardiac stem cell proliferation protein rose quickly from the time of injection to about day 8, after which it remained relatively constant at about 8 ng/ml over the course of the next 8 days. Cardiac stem cell proliferation protein delivered by osmotic pump as described above exhibited a relatively constant plasma concentration of between about 3 and 6 ng/ml over the course of 16 days (FIG. 14b). Adenoviral delivery of cardiac stem cell proliferation protein to pigs is shown in FIG. 15. Analysis of the MRI data scans obtained for pigs indicated that adenoviral cardiac stem cell proliferation protein administration reduced infarct volume size by about 21% as compared to 6% for control (FIG. 16), increased left ventricular mass by about 48% as compared to 28% for the control (FIG. 17), enhanced the left ventricular ejection fraction by 52% as opposed to the control which was reduced by about −4% (FIG. 18).

The present invention has been described with regard to one or more embodiments. However, it will be apparent to persons skilled in the art that a number of variations and modifications can be made without departing from the scope of the invention as defined in the claims.

The contents of all references and patents cited throughout this application are hereby incorporated by reference in their entirety.

The embodiments of the invention in which an exclusive property or privilege is claimed are defined as follows:

1. A cardiac stem cell proliferation protein comprising SEQ ID NO:1, a protein having at least 70% amino acid identity to SEQ ID NO:1, 2 or 3 or a fragment of a cardiac stem cell proliferation protein comprising
a) at least about 25 consecutive amino acids of SEQ ID NO:1, SEQ ID NO:2 or SEQ ID NO:3, or
b) a polypeptide having at least 70% amino acid identity to the fragment as defined in a); and;
wherein the cardiac stem cell proliferation protein or fragment thereof is not SEQ ID NO:2 or SEQ ID NO:3 and, wherein;
the cardiac stem cell proliferation protein or fragment thereof promotes proliferation and/or differentiation of cardiac stem cells.

2. A nucleic acid encoding the protein or fragment of claim 1.

3. A vector comprising the nucleic acid of claim 2.

4. A composition comprising the cardiac stem cell proliferation protein or fragment thereof according to claim 1 and a pharmaceutically acceptable carrier, diluent or excipient.

5. A kit comprising,
a) a cardiac stem cell proliferation protein or fragment thereof as defined by claim 1, or a nucleotide sequence encoding the cardiac stem cell proliferation protein or fragment thereof as defined by claim 1; and;
b) one or more pharmaceutically acceptable carriers, diluents, or excipients;
c) one or more devices for delivering or administering said cardiac stem cell proliferation protein, fragment thereof or a nucleotide sequence encoding said cardiac stem cell proliferation protein or fragment thereof;
d) instructions for administering said cardiac stem cell proliferation protein, fragment thereof or a nucleotide sequence encoding said cardiac stem cell proliferation protein or fragment thereof, or
e) any combination or sub-combination of b-d.

6. The vector of claim 3, wherein said vector is an adenoviral vector capable of expressing cardiac stem cell proliferation protein or a fragment thereof in vivo.

7. A method of treating a subject having heart disease or a heart defect, said method comprising administering a cardiac stem cell proliferation protein or a fragment thereof as defined by claim 1 to the subject in at least one single continuous low dose over a period of from about 1 to about 30 days.

8. The cardiac stem cell proliferation protein or fragment thereof of claim 1, defined by the amino acid sequence:
(SEQ ID NO: 1)
GPLGSSRREGSLEDPQTDSSVSLLPHLEAKIRQTHSLAHLLTKYAEQLLQ
EYVQLQGDPFGLPSFSPPRLPVAGLSAPAPSHAGLPVHERLRLDAAALAA
LPPLLDAVCRRQAELNPRAPRLLRRLEDAARQARALGAAVEALLAALGAA
NRGPRAEPPAATASAASATGVFPAKVLGLRVCGLYREWLSRTEGDLGQLL
PGGSA
(SEQ ID NO: 6)
GPLGSSRREGSLEDPQTDSSVSLLPHLEAKIRQTHSLAHLLTKYAEQLLQ
EYVQLQGDPFGLPSFSPPRLPVAGLSAPAPSHAGLPVHERLRLDAAALAA
LPP;
(SEQ ID NO: 7)
MSRREGSLEDPQTDSSVSLLPHLEAKIRQTHSLAHLLTKYAEQLLQEYVQ
LQGDPFGLPSFSPPRLPVAGLSAPAPSHAGLPVHERLRLDAAALAALPP;
(SEQ ID NO: 8)
GPLGSVCRRQAELNPRAPRLLRRLEDAARQARALGAAVEALLAALGAANR
GPRAEPPAATASAASATGVFPAKVLGLRVCGLYREWLSRTEGDLGQLLPG
GSA;
(SEQ ID NO: 9)
VCRRQAELNPRAPRLLRRLEDAARQARALGAAVEALLAALGAANRGPRAE
PPAATASAASATGVFPAKVLGLRVCGLYREWLSRTEGDLGQLLPGGSA;
(SEQ ID NO: 10)
GPLGSSRREGSLEDPQTDSSVSLLPHLEAKIRQTHSLAHLLTKYAEQLLQ
EYVQLQGDPFGLPSFSPPRLPVAGLSAPAPSHAGLPVHERLRLDAAALAA
LPPLLDAVCRRQAELNPRAPRLLRRLEDAARQARALGAAVEALLAALGAA
NRGP;
(SEQ ID NO: 11)
MSRREGSLEDPQTDSSVSLLPHLEAKIRQTHSLAHLLTKYAEQLLQEYVQ
LQGDPFGLPSFSPPRLPVAGLSAPAPSHAGLPVHERLRLDAAALAALPPL
LDAVCRRQAELNPRAPRLLRRLEDAARQARALGAAVEALLAALGAANRG
P;
(SEQ ID NO: 12)
GPLGSVQLQGDPFGLPSFSPPRLPVAGLSAPAPSHAGLPVHERLRLDAAA
LAALPPLLDAVCRRQAELNPRAPRLLRRLEDAARQARALGAAVEALLAAL
GAANRGPRAEPPAATASAASATGVFPAKVLGLRVCGLYREWLSRTEGDLG
QLLPGGSA;
(SEQ ID NO: 13)
VQLQGDPFGLPSFSPPRLPVAGLSAPAPSHAGLPVHERLRLDAAALAALP
PLLDAVCRRQAELNPRAPRLLRRLEDAARQARALGAAVEALLAALGAANR
GPRAEPPAATASAASATGVFPAKVLGLRVCGLYREWLSRTEGDLGQLLPG
GSA;
(SEQ ID NO: 14)
GPLGSVQLQGDPFGLPSFSPPRLPVAGLSAPAPSHAGLPVHERLRLDAAA
LAALPPLLDAVCRRQAELNPRAPRLLRRLEDAARQARALGAAVEALLAAL
GAANRGP;
(SEQ ID NO: 15)
VQLQGDPFGLPSFSPPRLPVAGLSAPAPSHAGLPVHERLRLDAAALAALP
PLLDAVCRRQAELNPRAPRLLRRLEDAARQARALGAAVEALLAALGAANR
GP;
(SEQ ID NO: 16)
GPLGSVCRRQAELNPRAPRLLRRLEDAARQARALGAAVEALLAALGAANR
GP;
(SEQ ID NO: 17)
VCRRQAELNPRAPRLLRRLEDAARQARALGAAVEALLAALGAANRGP;
(SEQ ID NO: 18)
GPLGSRAEPPAATASAASATGVFPAKVLGLRVCGLYREWLSRTEGDLGQL
LPGGSA;
(SEQ ID NO: 19)
RAEPPAATASAASATGVFPAKVLGLRVCGLYREWLSRTEGDLGQLLPGGS
A;
(SEQ ID NO: 20)
MSQREGSLEDHQTDSSISFLPHLEAKIRQTHNLARLLTKYAEQLLEEYVQ
QQGEPFGLPGFSPPRLPLAGLSGPAPSHAGLPVSERLRQDAAALSVLPA
L;
(SEQ ID NO: 21)
MSQREGSLEDHQTDSSISFLPHLEAKIRQTHNLARLLTKYAEQLLEEYVQ
QQGEPFGLPGFSPPRLPLAGLSGPAPSHAGLPVSERLRQDAAALSVLPAL
LDAVRRRQAELNPRAPRLLRSLEDAARQVRALGAAVETVLAALGAAARG
P;
(SEQ ID NO: 22)
VRRRQAELNPRAPRLLRSLEDAARQVRALGAAVETVLAALGAAARGPGPE
PVTVATLFTANSTAGIFSAKVLGFHVCGLYGEWVSRTEGDLGQLVPGGV
A;
(SEQ ID NO: 23)
EPFGLPGFSPPRLPLAGLSGPAPSHAGLPVSERLRQDAAALSVLPALLDA
VRRRQAELNPRAPRLLRSLEDAARQVRALGAAVETVLAALGAAARGPGPE
PVTVATLFTANSTAGIFSAKVLGFHVCGLYGEWVSRTEGDLGQLVPGGV
A,
or
(SEQ ID NO: 24)
QQGEPFGLPGFSPPRLPLAGLSGPAPSHAGLPVSERLRQDAAALSVLPAL
LDAVRRRQAELNPRAPRLLRSLEDAARQVRALGAAVETVLAALGAAARG
P.
